(19) United States
(12) Patent Application Publication
(10) Pub. No.: US 2023/0264008 A1
(43) Pub. Date: Aug. 24, 2023

(54) Enteral Feeding System

(71) Applicant: Joseph P. Schultz, Atlanta, GA (US)

(72) Inventor: Joseph P. Schultz, Atlanta, GA (US)

(21) Appl. No.: 17/933,292

(22) Filed: Sep. 19, 2022

Related U.S. Application Data
(60) Provisional application No. 63/245,426, filed on Sep. 17, 2021.

(51) Int. Cl. A61M 39/10; A61M 39/22

(52) U.S. Cl. CPC A61M 39/10; A61M 39/22; A61M 2039/1077; A61M 2039/1083

(57) ABSTRACT

Enteral adapters disclosed herein allow connection to gastrostomy tubes without leakage and cleanliness issues that accompany conventional connectors. Enteral adapters disclosed herein can comprise valved openings to the fluid conduit, removable or retractable connection elements to facilitate easy and secure connections, and cleaning. Enteral feeding systems and methods are also disclosed herein.

### CROSS-REFERENCE TO RELATED APPLICATION

This application claims the benefit of U.S. Provisional Application No. 63/245,426, filed on Sep. 17, 2021, the disclosure of which is incorporated herein by reference in its entirety.

### BACKGROUND

There is a need for a better enteral feeding system that is not prone to the misconnections of the prior art systems that had led to accidental infusions of enteral feedings into the wrong lumen such as into an intravenous tubing with an ISO 594 Luer lock. Recently, a new global standard ISO 80369-3 was developed to address enteral feeding misconnections that we considered to be among the top 10 technology hazards found in hospitals, according to the Emergency Care Research Institute (ECRI).

These standard ISO 80369-3 connectors have been referred to as ENIFit® connectors. These connectors are “(m)ade to precise ISO 80369 standards established by experts around the world, they function as a system to prevent misconnections between devices that can result in patient injury or death. For instance, ENIFit® enteral feeding connectors do not allow connectivity with devices for other clinical uses. All ISO 80369 connectors are important because they function together as a system, not individual connectors.” However, there are disadvantages to these ISO 80369-3 enteral connectors of the ISO 80369 system; including poor accuracy in small dosing, absence of venting capabilities, the small opening of the connectors, and the lack of benefit of new ENIFit® connectors. Other disadvantages of the new ISO 80369-3 enteral feeding standard are the moat design which makes the devices hard to clean and creates unacceptable levels of bacteria. Another disadvantage of the new enteral standard is that ENIFit® connectors are unable to meet the needs of the home-feeding community that prefer a blenderized diet; because the connectors have a narrower opening than some prior systems, and the narrow opening is perceived to slow the feeding and can be more prone clogging.

Accordingly, enteral adapters, systems, and methods that address the disadvantages of conventional enteral feeding connectors and systems are desirable.

### SUMMARY

Enteral adapters are disclosed herein and can comprise a nutrition source connector, a gastrostomy tube connector, and a fluid conduit extending from the nutrition source connector to the gastrostomy tube connection. The nutrition source connector can be a push fit connector. In certain aspects, the enteral adapter can further comprise a circumferential wall surrounding at least a portion of the conical tip, wherein the circumferential wall is removable or retractable. In certain aspects, the nutrition source connector can comprise a valve configured to alternatively open and close the fluid conduit.

Enteral feeding systems are also contemplated herein, comprising any of the enteral adapters disclosed herein and a nutrition source configured to connect to the nutrition source connector of the enteral adapter. Systems contemplated herein also can comprise any or all of a cap for any connection of the enteral adapter, a cleaning solution, a carrying case, a gastrostomy tube connection, and a gastrostomy tube.

Methods for providing nutrition from a nutrition source into a gastrostomy tube are also disclosed herein, and can comprise connecting an enteral adapter to a gastrostomy tube, connecting the nutrition source to the enteral adapter, transferring a substance from the nutrition source to the gastrostomy tube through the enteral adapter, and disconnecting the enteral adapter from the gastrostomy tube. Methods disclosed herein may reduce leakage, bacterial growth and related infection, assist cleaning, and provide convenient and safe access compared to conventional methods.

### BRIEF DESCRIPTION OF THE FIGURES

FIG. 1A shows an example of a conventional enteral connector secured to a feeding tube, configured to connect directly to a nutrition source, and a schematically represented cleaning tool compressing debris into the moat of the connector.

FIG. 1B shows an example of a conventional enteral connector secured to a feeding tube, configured to connect directly to a nutrition source, and a schematically represented cleaning tool removing debris from the connector.

FIG. 2A shows an embodiment of an enteral adapter.

FIG. 2B shows another embodiment of an enteral adapter.

FIG. 2C shows an embodiment of an enteral adapter in operation with a gastrostomy tube.

FIG. 2D shows the embodiment of FIG. 2A with the circumferential wall in a retracted position.

FIG. 2E shows the embodiment of FIG. 2C, configured to operate with the embodiment represented in FIG. 2D.

FIG. 2F shows the embodiment shown in FIG. 2C in a retracted position.

FIG. 2G shows a schematic representation of the rotational freedom between the circumferential wall and enteral adapters.

FIG. 2H shows the circumferential wall as removed from an enteral adapter.

FIG. 3A, FIG. 3B, and FIG. 3C represent a system comprising an enteral adapter (FIG. 3A) and two removable circumferential walls (FIG. 3B and FIG. 3C) capable of being secured to the enteral adapter.

FIG. 3D and FIG. 3E represent the system shown in FIG. 3A, FIG. 3B, and FIG. 3C with one circumferential wall of the system secured to the enteral adapter (FIG. 3D), and a second circumferential wall (FIG. 3E) removed from the enteral adapter.

FIG. 3F represents the system shown in FIG. 3A, showing the alternate configuration of first and second removable circumferential.

FIG. 3G represents the system shown in FIG. 3A, showing the second circumferential wall removed from the enteral adapter.

FIG. 4A shows an embodiment of an enteral adapter comprising a twist-to-open valve.

FIG. 4B comprises a removable circumferential wall configured to secure to the embodiment shown in FIG. 4A.

FIG. 4C shows the removable circumferential wall of FIG. 4B secured to the enteral adapter shown in FIG. 4A.

FIG. 4D shows an embodiment of an enteral adapter with a twist-to-open valve where the circumferential wall has a closed end state.

FIG. 4E shows an embodiment of an enteral adapter with a twist-to-open valve where the circumferential wall has an open end state.

FIG. 5A shows an embodiment of an enteral adapter with a retractable circumferential wall and push-to-open valve in a closed state.

FIG. 5B shows the enteral adapter of FIG. 5A with the circumferential wall in a retracted position and the push-to-open valve in an open state.

FIG. 5C shows a system comprising the enteral adapter of FIG. 5A, a second circumferential wall, and a gastrostomy tube.

FIG. 5D shows the system of FIG. 5C, with the second circumferential wall secured to the enteral connector and the push-to-open valve in an open state.

FIG. 5E shows the system of FIG. 5D connected to the gastrostomy tube.

FIG. 6A shows a schematic interaction between the enteral adapter of FIGS. 5A-5B and a female connector of a nutrition source in an unsealed position.

FIG. 6B shows a schematic interaction between the enteral adapter of FIGS. 5A-5B and a female connector of a nutrition source in a sealed position, with push-to-open valve in a closed state.

FIG. 6C shows a schematic interaction between the enteral adapter of FIGS. 5A-5B and a female connector of a nutrition source in a sealed position with push-to-open valve in an open state.

FIG. 7A shows an embodiment of an enteral feeding system comprising a push-to-fit nutrition source and the enteral adapter shown in FIG. 3A.

FIG. 7B shows an embodiment of an enteral feeding system comprising a twist-to-fit nutrition source and the enteral adapter shown in FIG. 3A.

FIG. 8A shows a cross-sectional view of the threadless female coupling shown in FIG. 7A.

FIG. 8B shows a front view of the threadless female coupling shown in FIG. 7A.

FIG. 8C, FIG. 8D, FIG. 8E, and FIG. 8F each shows alternative embodiments of threadless female couplings compatible with certain enteral adapters disclosed herein.

FIG. 9A shows a front view of an enteral adapter comprising a projection to facilitate locking the connector.

FIG. 9B shows a back view of the enteral adapter of FIG. 9A, comprising a projection to facilitate locking the connector.

FIG. 9C shows a side view of the enteral adapter of FIG. 9A, comprising a projection to facilitate locking the connector.

FIG. 9D, FIG. 9E, FIG. 9F, and FIG. 9G show front, back, side, and cross-sectional views of an enteral adapter comprising a threadless circumferential wall, respectively.

FIG. 10 shows an enteral feeding system comprising two enteral adapters.

FIG. 11A shows another embodiment of an enteral feeding system or component thereof, comprising an enteral syringe and a circumferential wall configured to attach to the enteral syringe.

FIG. 11B shows the embodiment of FIG. 11A, with the circumferential wall attached to the enteral syringe and positioned about an enteral adapter.

FIG. 11C shows the embodiment of FIG. 11B, with the circumferential wall attached to the enteral syringe and advanced along the female connector of an enteral syringe, the enteral syringe positioned about an enteral adapter.

FIG. 11D shows the embodimemt of FIG. 11C after subsequent push-fit of the female connector of the enteral syringe to the enteral adapter, and twist-fit of the circumferential wall to the enteral adapter.

FIG. 12A is a reproduction of the embodiment shown in FIG. 11D for reference.

FIG. 12B shows the enteral feeding system of FIG. 11A with the enteral syringe removed from the enteral adapter and circumferential wall.

FIG. 12C shows the enteral adapter of FIG. 11A with a cap.

FIG. 12D shows the enteral adapter of FIG. 11A with the cap secured to the enteral adapter.

FIG. 13 shows another enteral feeding system comprising an enteral adapter, an enteral syringe, and a circumferential wall provided as either of an independent component of the system or as attached to the enteral syringe.

FIG. 14 shows an enteral feeding system comprising an enteral adapter configured to attached to a gastrostomy tube via external threadings, a push-fit cap without internal threading, a twist-fit cap with internal threading, and a circumferential wall

FIG. 15 shows an enteral feeding system comprising an enteral adapter configured to attach to a gastrostomy tube via mic-key configuration as presented in FIG. 9A, a push-fit cap without internal threading, a twist-fit cap with internal threading, and a circumferential wall.

### Definitions

To define more clearly the terms used herein, the following definitions are provided. Unless otherwise indicated, the following definitions are applicable to this disclosure. To the extent that any definition or usage provided by any document incorporated herein by reference conflicts with the definition or usage provided herein, the definition or usage provided herein controls.

Enteral adapters disclosed herein generally can be compatible with a male ISO 80369-3 connection of a gastrostomy tube, while not necessarily complying with the standard in all aspects. Similarly, enteral adapters contemplated herein also can generally comprise a connection to a nutrition source that can be ISO 80369-3 compatible or ISO 80369-3 compliant.

Connections of the enteral adapter generally can be connected by a fluid conduit that allows transfer of a nutritious substance from a nutrition source into a gastrostomy tube. Generally, the fluid conduit can be understood to be in fluid communication with at least an entry and exit of enteral adapters disclosed herein, such as may allow efficient transfer of a nutritious substance through the enteral adapter. The fluid conduit can therefore be a hollow interior space of the adapter terminating at the nutrition source connector at one end and the enteral connector at a second end. The fluid conduit also may be branched to accommodate enteral adapters with more than one enteral connector or nutrition source connectors. The fluid conduit generally can be referred to herein as being in either of an open, closed, or intermediate position and will be understood to refer to the capacity in which the fluid conduit is in fluid communication with a nutrition source or enteral connector. For instance, fluid can be delivered to the fluid conduit at the nutrition source connector when the fluid conduit is in an open state, but not in a closed state. The state of the fluid conduit may be considered independent from the state of an associated connection to the enteral adapter. For instance, in certain aspects, a nutrition source may be completely or partially connected to the nutrition source connector, but the fluid conduit may remain in a closed state such that the nutrition source is not in fluid communication with the fluid conduit.

As used herein, nutrition source and nutritious substances will be understood to apply generally to any substance that may be applied within an enteral feeding system. Nutritious substances as contemplated herein can comprise liquids, solids, or slurried mixtures. Accordingly, it will also be appreciated that references to components in “fluid communication” can refer to a state where the transfer of solids and slurried mixtures can be possible, as well as fluids. Nutritious substances therefore can refer to any combination of foods, medicines, and fluids that may be advantageously delivered to the enteral system via a gastrostomy tube.

Connections of enteral adapters disclosed herein are contemplated as comprising a push fit connection in certain aspects. As used herein, push fit connections will be understood to encompass connections configured to form a secure and sealed connection between male and female connectors without twisting. Simply, a push fit connection may be securely connected by pushing two connectors together (e.g., along a shared axis of the connectors) without further action to lock the connection or open a fluid conduit in communication with the connectors. In this sense, push fit connectors may be seen as complementary to twist fit connectors, that may require rotation of one or both connectors about the axis of the connection being made. It will be appreciated that twist-fit connectors described herein also may include push fit component along the axis of the connection.

References to ENFIT® connectors made herein generally are understood to be compliant with the ISO 80369-3 enteral connectors. ENFIT® connectors as referred to herein will be understood to represent a specific example of an ISO 80369-3 compliant enteral connector. Other arrangements compliant with the ISO 80369-3 standard are also contemplated herein. Connectors that are not compliant with the ISO 80369-3 standard are also contemplated herein, and are demonstrated in certain aspects to have advantages over ISO 80369-3 compliant connectors such as represented by ENFIT®. Thus, connectors can be compatible with the ISO 80369-3 standard without complying with the 80369-3 standard. Alternatively, connectors disclosed herein can be neither compatible nor compliant with the ISO 80369-3 standard.

Similarly, components of enteral adapters may be described herein as removable or retractable. For instance, certain aspects of enteral adapters can comprise a circumferential wall that is removable from the adapter. It is intended that removable features described herein can be detached from the adapter completely to provide separate components. Separated components will be understood as able to be reattached without causing damage to either component or requiring special knowledge or tools to remove and reattach the component. Certain aspects may comprise retractable components, which will be understood to be distinct from removable components, though retractable components may also be removable. Retractable components as used herein can be any that may be at least partially compressed, repositioned, relocated, withdrawn, etc. from a first position to a second position while being retained on the adapter (e.g., without being completely removed from the adapter).

In general, an amount, size, formulation, parameter, range, or other quantity or characteristic is “about” or “approximately” whether or not expressly stated to be such. Whether or not modified by the term “about” or “approximately,” the claims include equivalents to the quantities or characteristics.

### DETAILED DESCRIPTION

Enteral adapters and systems and methods of use are disclosed herein as improvements to the convenience, cleanliness, efficiency and ease-of-use of conventional enteral feeding systems.

### Enteral Adapters

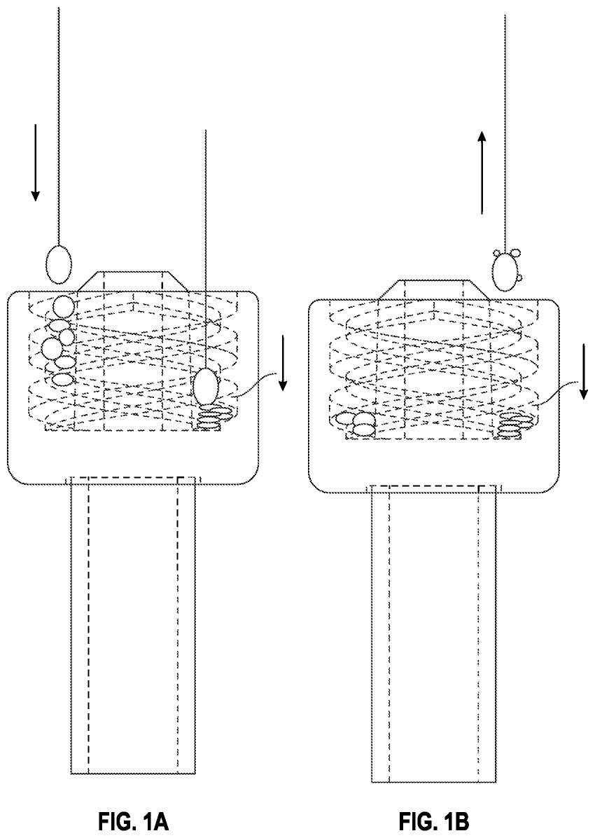

Enteral adapters contemplated herein generally can comprise a nutrition source connector, a gastrostomy tube connector, and a fluid conduit extending from an opening in the nutrition source connector to an opening in the gastrostomy tube connector. Generally, enteral adapters disclosed herein can be compatible with enteral connectors common to gastrostomy tubes, such as is depicted in FIG. 1. FIG. 1 shows a representation of an enteral connector of the prior art. FIG. 1A shows how food can be trapped within the “moat” of the connector. A schematically represented cleaning tool such as a swab is inserted into the moat, to clean it; however, the downward motion of the swab compresses the debris deeper into the moat making it harder to retrieve. FIG. 1B shows a simple swab represented as a cleaning tool to remove the debris. However, the insertion of the cleaning tool can further compact the debris deeper into the crevice. Debris on the top of the swab plug gets pulled out of the moat, but debris below the plug may be left behind.

Moreover, enteral syringes generally may be more prone to leakage and contamination than luer tip syringes of the prior art due to their larger openings to accommodate transfer of thicker fluids than might be introduced via an IV line, for instance. The maximum sizes of the fluid openings of female connectors of ISO 80369-3 enteral syringes include an inside diameter at the open end of the female taper of 5.74 mm (nominal dimension of 5.69 mm+/−0.05 mm), and an inside diameter of the fluid lumen of the connector of 2.95 mm (nominal dimension of 2.90 mm+/−5 mm) at the opposite end of the female connector. By comparison, in the ISO 80369-7 specification for an ISO 80369-7 male luer slip connector for connectors for intravascular or hypodermic applications dictates a commonly used nominal diameter of 2.10 (and a 2.90 mm maximum dimension). The standard dimensions of ISO 80369-3 enteral syringes, therefore makes them even more prone to leakage when oriented in tip downward position, i.e., during the requisite positioning and twisting actions required prior to forming a seal; when compared to other slip connectors devices such as male luer slip syringe connectors of the prior art. The internally threaded collar design of the ISO 80369-3 enteral syringes built into the ISO 80369-3 specification to improve securement for long term feeds, therefore may cause new previously unidentified risks for increased leakage due to the relatively larger openings; plus the additional manipulation required during connection and prior to sealing. Leakage can lead to increased contamination issues due to debris trapped in the moat.

Furthermore, in comparison to a typical male luer lock delivery syringe comprising a small opening and a threaded moat, it is of little consequence should the threaded collar become contaminated as the typical male luer lock delivery syringe will typically be thrown away. On the other hand, the use of ISO 80369-3 having conical male connectors with surrounding threaded collars is oriented on the receiving end of an indwelling gastrostomy tube. An indwelling gastrostomy tube is not single use and not disposable. The feeding solutions are generally thicker and more nutrient rich than IV fluids. The “female” needleless access valves on the receiving end are relatively easy to clean off. Whereas moat retention issues of threaded collars on luer locks of single use delivery devices delivering IV solutions are generally overlooked; the significance of these issues is much greater when used on a large scale with multiple use in-dwelling devices on the receiving end of thick, particulate, nutrient rich feeding solutions on the receiving end.

Enteral adapters contemplated herein prevent leakage and debris from accumulating within this moat and provide more accessible cleaning when the adapter is soiled.

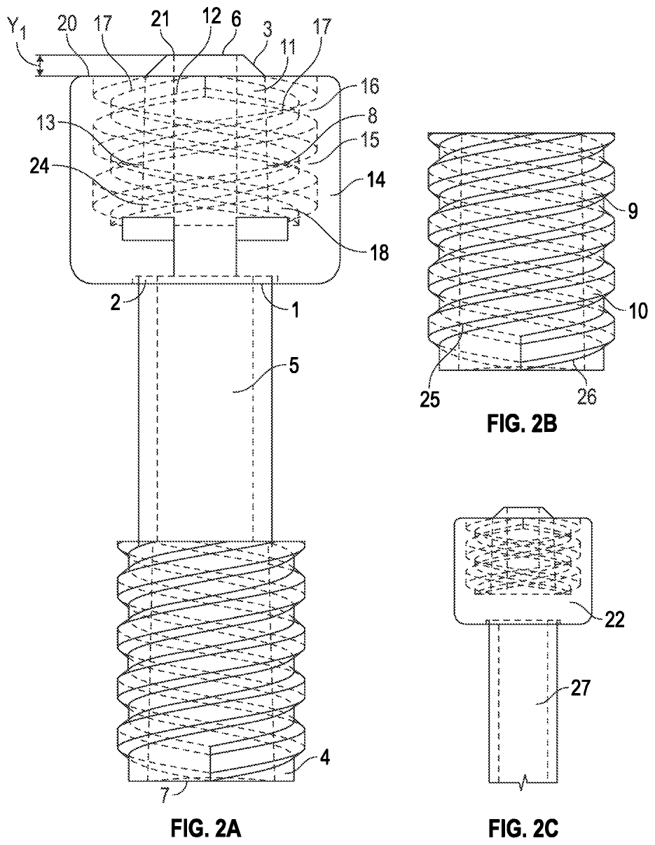

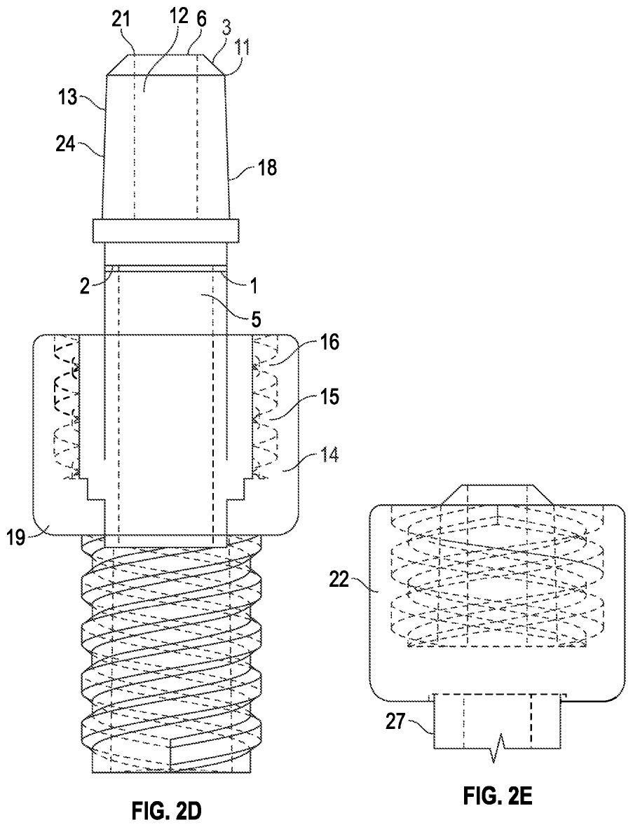

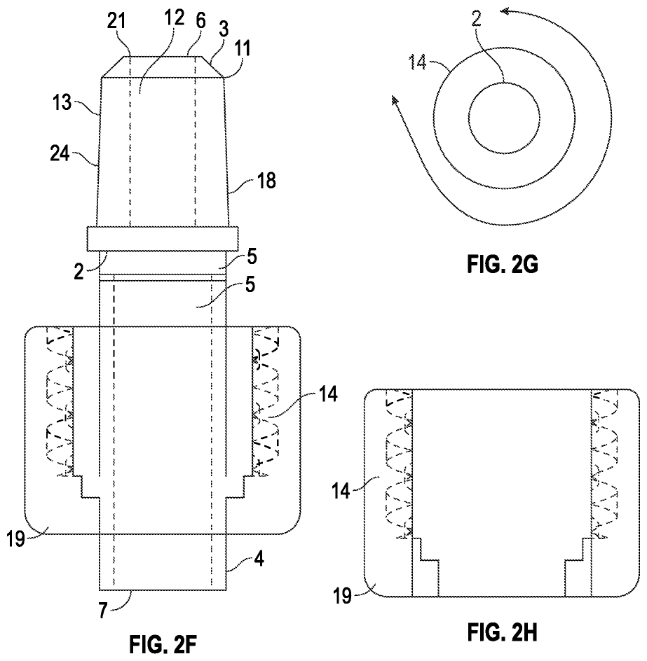

For instance, FIG. 2A provides a first embodiment of an enteral adapter 1 within the scope of this disclosure, comprising nutrition source connector 3, gastrostomy connector 4, and a fluid conduit 5 extending therebetween. In certain aspects, the fluid conduit can extend to an opening in the nutrition source connector (e.g., the distal end of the conical tip, a tapered surface of the conical tip). As shown in FIG. 2A, fluid conduit 5 is shown extending from a distal end 6 to a proximal end 7 comprising an inner surface 12 and an outer surface 13 of fluid conduit wall 11, defining an internal space of the fluid conduit for the passage of the nutrition source.

The nutrition source connector 3 generally can be configured to connect to any nutrition source. For instance, outer surface 13 may be configured to at least partially interface with the nutrition source as part of a male conical tip 24. In certain aspects, the nutrition source connector can be an ISO 80369-3 compliant or compatible connector. In other aspects, the nutrition source connector can be incompatible with a standard female Luer connector. Generally, the nutrition source connector can be any that afford advantages to the system as described herein, particularly cleanliness, convenience, ease-of-use, and while maintaining safety by distinction from IV connectors. The nutrition source connector generally may be any combination of features that provide a secure connection between the fluid conduit and a particular or general nutrition source.

In certain aspects the nutrition source connector can comprise a push fit connector. In certain aspects, the push fit connection can comprise a conical tip, e.g., a male conical taper, the conical tip configured to connect with a nutrition source comprising a compatible female conical recess. For instance, male conical taper 24 of FIG. 2A comprises a coupling surface 8 that is shaped within a conical recess of a compatible female adapter, such as conical recess 25 shown in FIG. 2B.

In certain aspects, the enteral adapter can comprise a circumferential wall either as part of the nutrition source connector as shown in FIG. 2A, wherein circumferential wall 14 partially surrounds conical tip 24 to provide internal threadings 15, 16 for a twist connection with a nutrition source (or further enteral adapters). The circumferential wall can be positioned in a connection position to form a gap 17 between the wall and the nutrition source connector 3. Alternatively, a circumferential wall may be provided independent from the function of the nutrition source connector to form a connection with a nutrition source, and merely surround a portion of the connector. For instance, the circumferential wall can comprise (or consist of) a collar and have an open end 20 that partially surrounds male conical tip 24 of the nutrition source connector. As a result, the distal end of male conical tip 21 may extend axially beyond the circumferential wall 14 a distance Y1. In certain aspects, Y1 may be in a range from 0.5 to 2 mm, or about 1.2 mm. In certain aspects, Y2 may therefore be greater than 2 mm, greater than 1.2 mm, or in a range from 1.2 to 5 mm.

For example, as shown in FIG. 2A, nutrition source connector 3 can be a twist connector comprising conical tip 24 paired with a circumferential wall 14 comprising internal threadings 15, 16 configured to mate with a threading of a nutrition source or a further enteral adapter. For instance, second enteral adapter 9 shown in FIG. 2B may be connected to the nutrition source connector of an enteral adapter, e.g., nutrition source connector 3 of enteral adapter 1 shown in FIG. 2A. Enteral adapter 9 of FIG. 2B can comprise a conical recess 25 capable of receiving conical tip 24 of FIG. 2A, and external threads 26 configured to mate with an enteral feeding format, for example, threadings 15, 16 of internal threaded circumferential wall 14 represented in FIG. 2A. The embodiment of FIG. 2B also may be coupled by threaded coupling 10 to an ISO 80369-3 compliant enteral feeding format, such as is embodied by enteral connector 22 of FIG. 2C attached to feeding tube 27.

Other embodiments of the present invention may have variable distances of Y1 from the open end 20 of the collar to the distal end 21 of the first end 3 of the first enteral adapter base 2. The distance of Y1 may be equivalent to the distance between similar features of an ISO 80369-3 compliant connector, such as reference dimension “c” of Table B.1 of the ISO 80369-3 standard; or the distance Y1 may be greater than the largest allowable distance between similar features of an ISO 80369-3 compliant connector (max. dimension of “c” is 1.20 mm); and Y1 may be greater than 1.2 mm leading to greater exposure of the tip for easier access for inspection, maintenance and cleaning. The enteral adapter 1 may have a format compliant with an enteral connector standard, such as the ISO 80369-3 in some states (e.g., with a collar, as shown in FIG. 2A) and non-compliant in another state (e.g., without a collar, as shown in FIG. 2B).

In certain aspects, enteral adapter 1 may be configured to connect to a conventional enteral connector of a gastrostomy tube, as shown by FIG. 2C. As shown, the external threads of gastrostomy connector 4 can be configured to mate with internal threads of enteral connector 22, permanently secured to gastrostomy tube 27. Generally, enteral adapters contemplated herein can comprise a gastrostomy tube connector compatible or compliant with industry standard regulations, to allow more universal application of the enteral adapter to existing standards. In certain aspects, the gastrostomy connector can be an ISO 80369-3 compliant or compatible connector, such as an ENFIT® connector. However, alternative configurations of the gastrostomy tube connector that may be compatible with non-standard enteral connections and those yet to be developed are also contemplated herein.

In certain aspects, the gastrostomy connector can comprise a coupling surface for connection to an enteral feeding coupling format; a coupling with an ISO 80369-3 compliant format; an ISO 80369-3 compliant coupling; a coupling that is not an ISO 80369-3 compliant coupling; a coupling that is incompatible for mating to an ISO 80369-7 connector; a coupling that is an ISO 80369-3 compliant coupling and is not an ISO 80369-7 coupling; a coupling that is not an ISO 80369-3 compliant coupling and is not an ISO 80369-7 coupling; a conical coupling surface for connection to an enteral feeding coupling format; a conical coupling surface comprising conical surface dimensions of an ISO 80369-3 conical connector; a conical coupling surface comprising conical surface dimensions of an ISO 80369-3 conical connector and the conical connector surface dimensions consist of conical surface dimensions of an ISO 80369-3 conical connector; a recessed coupling surface for connection to an enteral feeding coupling format; a recessed coupling surface comprising conical surface dimensions of a recessed ISO 80369-3 conical connector; a threaded coupling surface for connection to an enteral feeding coupling format; an externally threaded coupling surface for connection to an enteral feeding coupling format; an externally threaded coupling surface capable of mating with a coupling of an ISO 80369-3 enteral connector; an externally threaded coupling surface comprising thread dimensions of an ISO 80369-3 connector; an externally threaded coupling surface comprising thread dimensions of an ISO 80369-3 connector that consist of thread dimensions an ISO 80369-3 connector; a coupling surface that is incompatible for mating to an ISO 80369-7 connector; a coupling surface that is capable of mating to an ISO 80369-3 connector and the second end comprises a coupling that is incompatible for mating to an ISO 80369-7 connector; an externally threaded coupling surface is incompatible for mating to an ISO 80369-7 connector; a conical coupling surface is incompatible for mating to an ISO 80369-7 connector; a recessed conical coupling surface is incompatible for mating to an ISO 80369-7 connector; a tapered connector, an externally tapered connector; a tube; a gastrostomy tube; a gastrostomy tube positioned within a patient; a syringe; a reservoir; and/or a coupling.

In certain aspects, circumferential walls as contemplated and disclosed herein may be removable or retractable from their position relative to the nutrition source connector, or portions thereof. Circumferential walls may comprise one or more internally threaded portions, or be entirely internally threaded. In certain aspects, the internal threads of a circumferential wall can extend at least from a distal portion of the circumferential wall to the base of the conical tip when secured to the adapter. In other aspects, at least a portion of the internal threads of the circumferential wall can be configured to mate with external threads of the enteral adapter (e.g., the nutrition source connector, the adapter base). For instance, the nutrition source connector can comprise an externally threaded surface proximal to, and/or extending from, the base of the conical tip such that the internal threads of the circumferential wall may be secured to the external threads of the nutrition source connector. Those of skill in the art will appreciate that the enteral adapter base and nutrition source connector may comprise positional restraints for advancing and retracting the circumferential wall thereabout. For instance, certain aspects the enteral adapter or nutrition source can comprise a partition axially projecting from the enteral adapter to provide a set an operational axial position of a removable or retractable circumferential wall when secured to the enteral adapter in operating position.

Circumferential walls as referred herein can be configured to removably secured to the adapter, and to removably secure a female connector of a nutrition source. Circumferential walls contemplated herein may be removed; retracted and rotated; retracted and removed; rotated and removed; or rotated, retracted and removed.

Referring to FIG. 2D, it is seen that circumferential wall 14 may be retracted from a connection position partially surrounding nutrition source connector 3 and male conical tip 24, to a retracted position, allowing enteral adapter 1 to accommodate a push fit connection and facilitate cleaning of the male conical tip after use of the enteral adapter. In the retracted position, the circumferential wall may be retained by interaction between the proximal end of the circumferential wall and an axially protruding feature of the enteral adapter, such as external threadings of the gastrostomy tube connector 4. In certain aspects, the connection the retractable or removable circumferential wall may be retained in a connected position by biasing a proximal end of the circumferential wall against generally any suitable axial projection from the enteral adapter body 2. In certain aspects, enteral adapters may comprise a circumferential wall as part of a retractable, push-to-open valve. In such aspects as described below, the circumferential wall can be retractable from a closed position adjacent a fluid conduit opening in a tapered surface of the male conical tip, blocking flow from entering the fluid conduit, to an open position. In certain aspects, the closed position of the circumferential wall can be distal to the open position. In other aspects, the circumferential wall can be attached to the enteral adapter by a compressible silicon sleeve. Such aspects may further comprise a second circumferential wall surrounding the nutrition source connector, which may also be removeable.

In this manner, gastrostomy connector 4 may remain accessible and able to be connected to an enteral feeding tube as represented by FIG. 2E, with the circumferential wall 14 positioned in either a connected position at least partially surrounding the nutrition source connector, or a retracted position retained about the axis of the fluid conduit 5.

In still further aspects, the circumferential wall 19 may adopt a retracted position as shown in FIG. 2F, or able to be removed completely from the adapter, as shown in FIGS. 2G and 2H. FIG. 2F shows the circumferential wall 14 that is retracted proximally from the distal end 21 of the first end 3 of the first enteral adapter base 2. Furthermore, the collar itself may be interchangeable with at least one other second collar. FIG. 2G schematically represents on end, a circumferential wall 14 that can rotate about the axis of a first enteral adapter. FIG. 2H shows a collar with a circumferential wall that is free of the first enteral adapter base and does not surround the enteral adapter base. This wall 14 may be part of a first collar that will be applied to surround a first enteral adapter base; part of a first collar that has been removed from surrounding a first enteral adapter base, part of at least a second collar that will be applied to surround a first enteral adapter base; or part of at least a second collar that has been removed from surrounding a first enteral adapter base.

In this manner, the enteral adapter and its components can be more easily cleaned or replaced following use, to maintain a sanitary system. As shown by FIGS. 2G-2H, certain aspects can comprise multiple interchangeable circumferential walls that can be removed and replaced, offering further flexibility to enteral feeding methods and maintenance of enteral adapters in a sanitary state. Again, enteral adapters as disclosed herein can have the advantage of being less prone to having the buildup of contaminants in the moat between a coupling surface 8 and circumferential internally threaded wall 14 (e.g., gap 17, the proximal portion of gap 17), since the moat can be abolished with the removal of the threaded collar providing better access for cleaning of the coupling surface 8 and the inner surface of the circumferential wall 14 and its threading 15.

Aspects comprising a removable collar with a removable wall can reduce the contaminants in the gap 17 and eliminate the need to employ mechanical cleaning with a specialized cleaning brush of the internal threading as conventionally required. A simple wipe or solution could be more successfully used with the more exposed couple surface. As a result, the need for purchasing, stocking and using the specialized prior art brushes and cleaning devices of the prior art and more successful cleaning is expected with specialized brushes or with more commonly available products will be reduced.

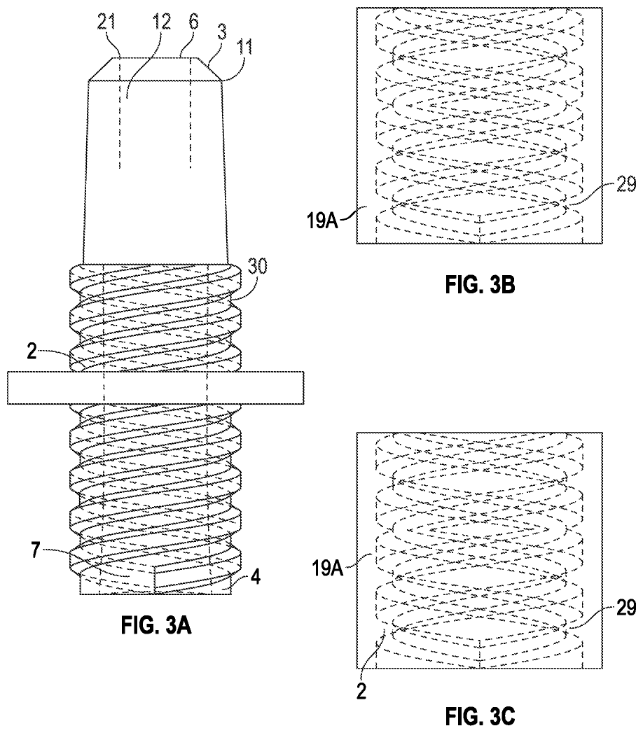

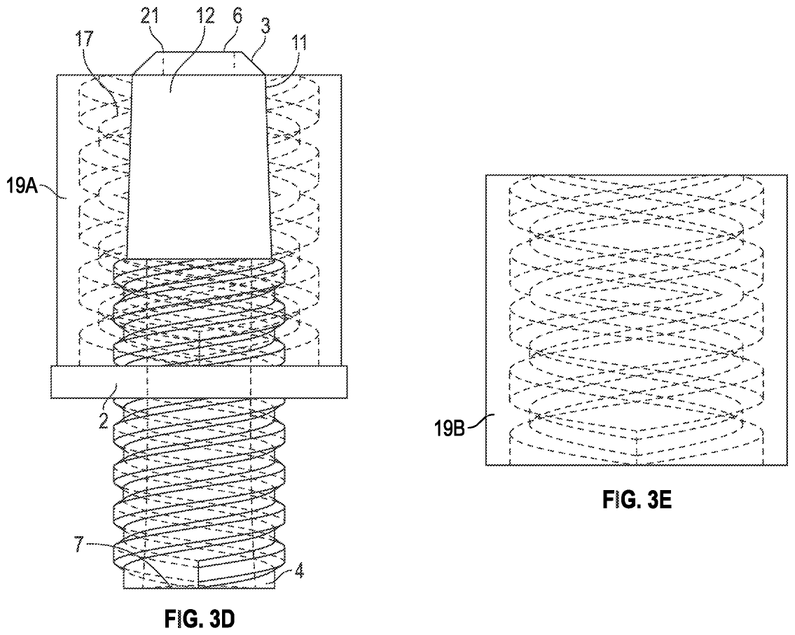

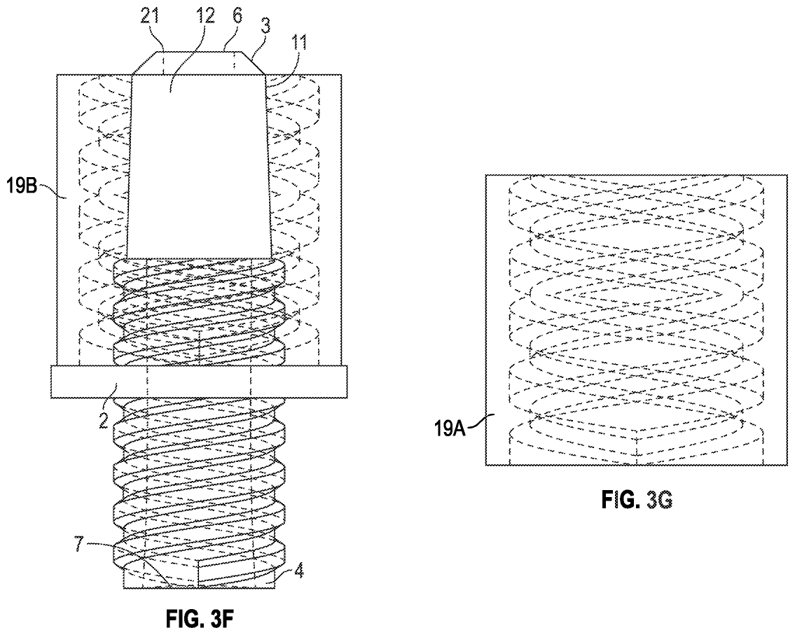

Enteral adapters disclosed herein also can comprise a circumferential wall that can be removed or retracted in a distal direction, or from the nutrition source connector. FIGS. 3A-3C represent embodiments of the enteral adapter comprising removable circumferential wall 19A, wherein wall 19A comprising internal threads 29 as a coupler for mating with external threads 30 of enteral adapter as shown in FIG. 3A. External threads may be compliant or compatible with ISO 80369-3 or any other appropriate standard. As above, multiple circumferential collars are shown to demonstrate interchangeability of the enteral adapter and its components, offering improved flexibility in maintaining an enteral feeding system in a sanitary condition. FIGS. 3D and 3E show the enteral adapter and circumferential walls 19A and 19B as a system where two circumferential walls are provided and interchangeable.

An example of the operation of an embodiment of this invention provides a first collar such as the collar 19A in FIG. 3B which may be attached to a first enteral adapter base, such as first enteral adapter base 2 in FIG. 3A to form the adapter configuration shown in FIG. 3D. When contaminates become trapped in the gap 17, the collar 19A may be removed to become a free collar as can be shown by the collar 19A in FIG. 3G. The naked conical connector can then be more easily accessed and cleaned with a simple cleaning wipe. The collar 19A might be cleaned or alternatively, a second collar, such as the collar 19B in FIG. 3E may be assembled to the first enteral adapter base. In addition to the exposure benefits of the tip, this collar exchange process would provide a new clean inner collar surface without the need to clean the difficult to reach inner collar surface.

Removable or retractable circumferential walls as described above also provide the option of selecting between a quicker access and faster slip fit engagement and disengagement and without spilling or leakage of fluid, and an alternative twist fit engagement as may be preferred where twist valves are employed. In still further aspects, removable or retractable collars may allow a twist fit connection to be applied after, and independent from when an initial push fit connection to a nutrition source is made. In this manner, the advantages of reduced leakage and a quick connection realized by push fit connections could also enjoy a secure connection with tighter and prolonged unmonitored use typically observed in a twist fit connection.

In certain aspects, the fluid conduit can comprise one or more valved openings configured to alternately open and close the opening in the nutrition source connector to the fluid conduit. In certain aspects, the valve can be configured to move from a closed position to an open position by connection of a nutrition source to the nutrition source connector.

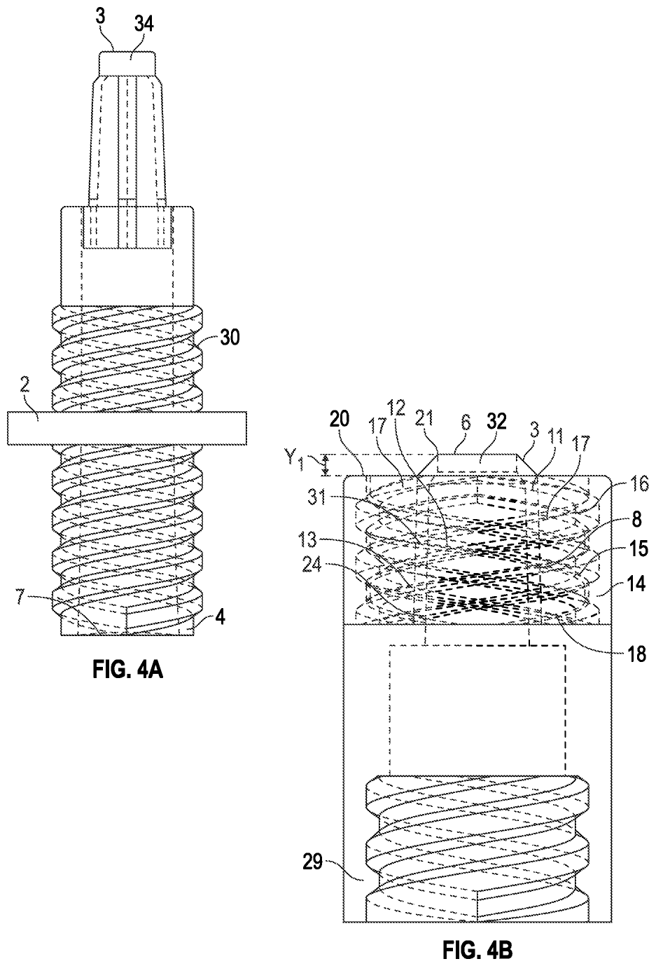

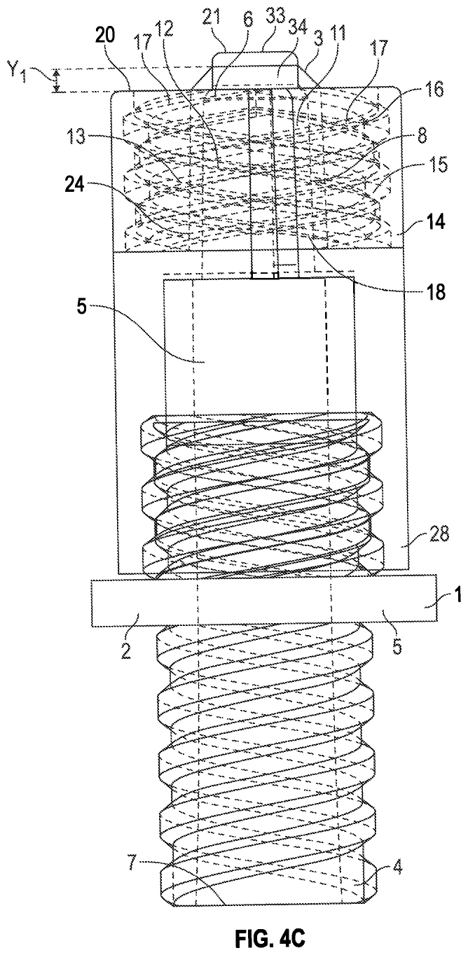

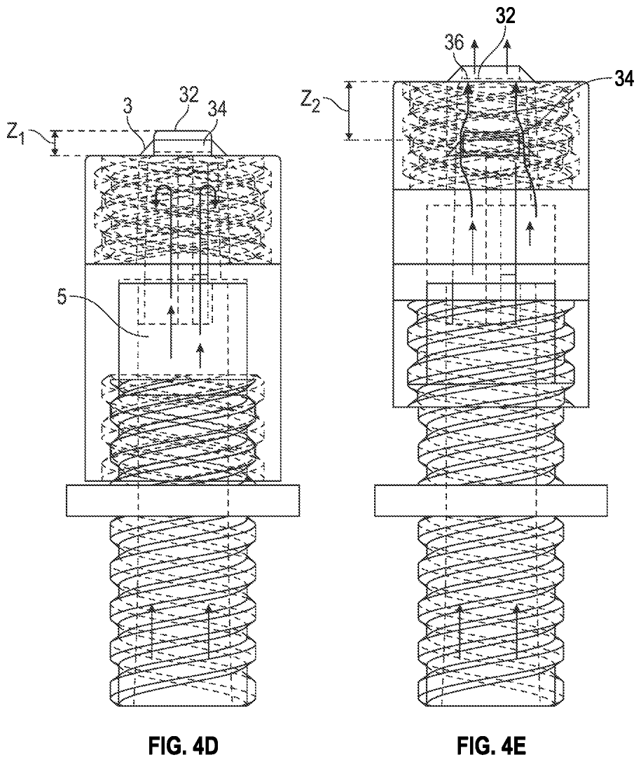

FIGS. 4A-4E provide embodiments of enteral adapters comprising a twist-to-open valve. As shown in FIG. 4A, the distal end of the fluid conduit can be terminated by a solid center wall 34 of a stem, the stem extending axially from the conical tip when the valve is moved from a closed position to an open position, and upon rotation of the conical tip relative to the stem. In certain aspects, a circumferential wall may be provided and configured to engage the valve, to open the valve upon twist-fit connection of a nutrition source. In certain aspects, a circumferential wall may be provided to the enteral adapter in any manner disclosed herein, and may interact with the fluid conduit valve upon connection, or via the nutrition source connector. FIG. 4B provides an embodiment of a circumferential to complement the valved enteral adapter shown in FIG. 4A, with internal threadings 29 configured to mate with external adapter threading 30 and be advanced into a connection position against the enteral adapter base 2.

As for other aspects, the circumferential wall may be shaped and configured to form a connection with a nutrition source (e.g., ISO 80369-3 compliant connection, an ISO 80369-3 compatible connection, an ISO 80369-7 connection). FIG. 4B provides an embodiment of a circumferential wall comprising a conical wall 31 with open end 32 allowing axial travel of the valve stem as described above. Conical wall 31 also comprises a tapered surface able to mate with a conical recess of a nutrition source.

Certain embodiments may have a fluid conduit 5 with a closed end. As shown in FIG. 4C, embodiments of the new enteral adapter coupling system may have a first end 3 comprising a closed end 33 with a solid center wall 34 for ease of cleaning of the more distal portions of the first end 3, to create a valve, to assist in forming a larger port in a side wall than at that tip or for other advantageous purposes. The first end 3 may have a closed end state as illustrated in FIGS. 4C and 4D and an open ended, as illustrated in FIG. 4E, for better control of flow during infusion, withdrawal, disconnection, maintenance, and storage. The center of the tip of the first end 3 may have solid tip end with a solid center wall 34 to prevent fluid from flowing through the center of the tip 35 of the first end 3. The tip may comprise a valve for improved regulation of fluid flow during connection and disconnection. For example, the valve may be open, as may be represented by the valve in FIG. 4E, during connection to a fluid source (such as a second enteral adapter that may be the second enteral adapter 9 of FIG. 2B). The same valve may also be closed when disconnected from a fluid source, as may be represented by the valve shown in FIG. 4D. This configuration can eliminate the need for an additional cap, as required in the prior art, as these caps can get lost leading to choking hazards and leakage hazards. Another advantage of the valved configuration is to eliminate need for a tethered cap that can get tangled on other medical apparatuses and which could then lead to dislodgement of the tube or could cause irritation when compressed against a skin surface. Furthermore, such a valve can reduce the operation steps to open and close the flow compared to capped devices relative to existing enteral adapters.

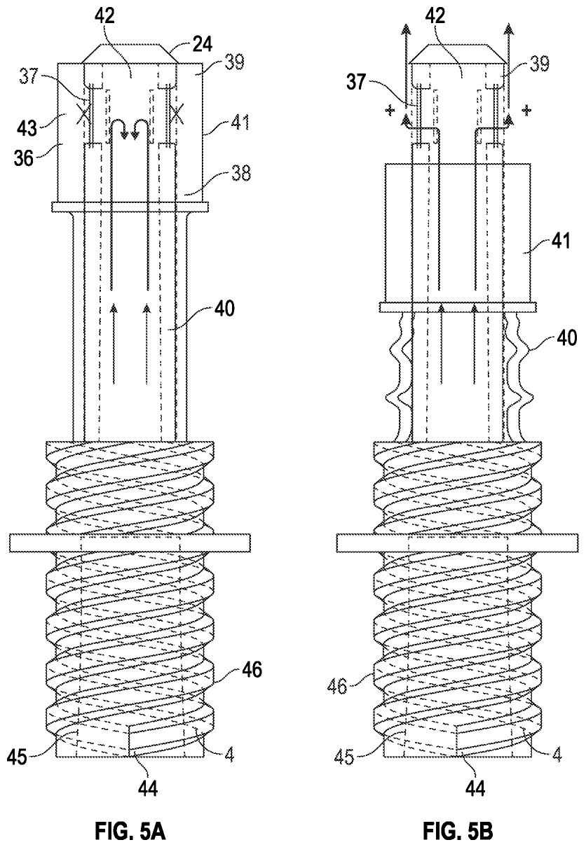

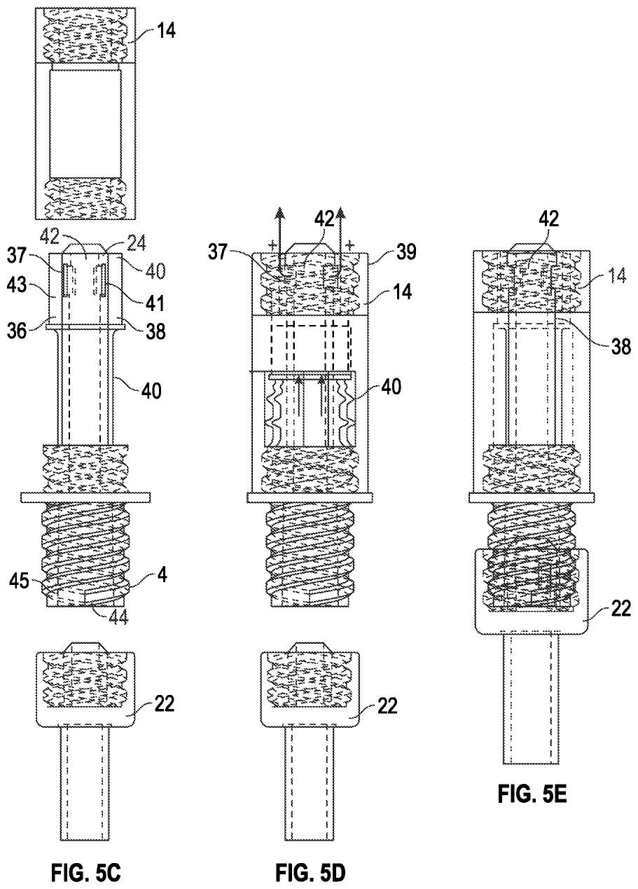

Enteral adapters disclosed herein also can comprise push-to-open valves. As above such valves may be configured to open upon connection of a nutrition source, or connection of a circumferential wall. FIGS. 5A and 5B illustrate an embodiment of an enteral adapter with a retractable valve in a closed state and open state, respectively. As illustrated, valved fluid conduit can comprise a conical tip 24 and a valve 36 formed in adjacent contact with the outer surface of the conical tip 24. The fluid conduit 5 may be terminated at the distal end by a closed end 42 (e.g., dead head conduit) and comprise at least one side opening 37. Compressible and retractable push-to-open valve 38 can comprise a wall 43 surrounding the side opening 37 at least at exit hatch 41 when the valve 36 is in a closed, uncompressed state as shown in FIG. 5A. In certain aspects, the wall can be secured to the enteral adapter base by a compressible sleeve 40, allowing compression of the sleeve in an axial direction along the enteral adapter, withdrawing the valve wall 43 to an open state, as shown in FIG. 5B. In certain aspects the compressible sleeve can comprise a silicon sleeve.

The valve embodiments shown in FIGS. 5A and 5B may be employed as shown in a “naked” configuration, without a surrounding circumferential wall. Such embodiments can be easier to clear then moated designs, and can provide quick connection and disconnection for short flushes of the connectors. Push-to-open valves as shown may also provide a seal without an integral twisting step that can result in leakage. Though embodiments may have a variety of connectors as the gastrostomy connector, the embodiments of FIGS. 5A and 5B are shown with a threaded connector that may be compatible with an ISO 80369-3 compliant connector, so the valved function can easily be retrofitted onto existing connectors. Alternative arrangements of the gastrostomy connector are also contemplated and disclosed herein.

Enteral adapters with push-to-open valves may further be accompanied by a twist fit connection. For instance, FIGS. 5C-E illustrate the embodiment of FIGS. 5A-5B incorporated into an enteral adapter feeding system as in operation with a conventional gastrostomy tube and connection 22. FIG. 5C shows a circumferential wall 14 that can be fitted over the valved connector. Also shown is a separate third enteral connector, that may be an ISO 80369-3 enteral connector. FIG. 5D shows a circumferential wall 14 that has been assembled over the valved connector, but not assembled to feeding tube connector 22. FIG. 5E shows the assembly of the valved connector onto the feeding tube connector 22, which can now function with the conventional enteral connector system and impart the new benefits and advantages of the valved adapter without replacing the conventional feeding tube connector 22 and without replacing the entire enteral feeding tube system.

For instance, in certain aspects, the circumferential wall 14 can provide compression force upon connection to the enteral adapter to operate the valve into an open state as shown in FIG. 5D. Such aspects may be aided using a second enteral adapter as embodied by FIG. 2B, such that the second enteral adapter can be connected between the circumferential wall 14 and a nutrition source prior to securing the circumferential wall 14 to the enteral adapter. Alternatively, the nutrition source may be connected directly to the circumferential wall 14 prior to connection of the circumferential wall to the first enteral adapter. In this manner, connection of circumferential wall to the enteral adapter can open fluid communication between the nutrition source and the enteral adapter within a closed system to prevent leakage at the valved connection.

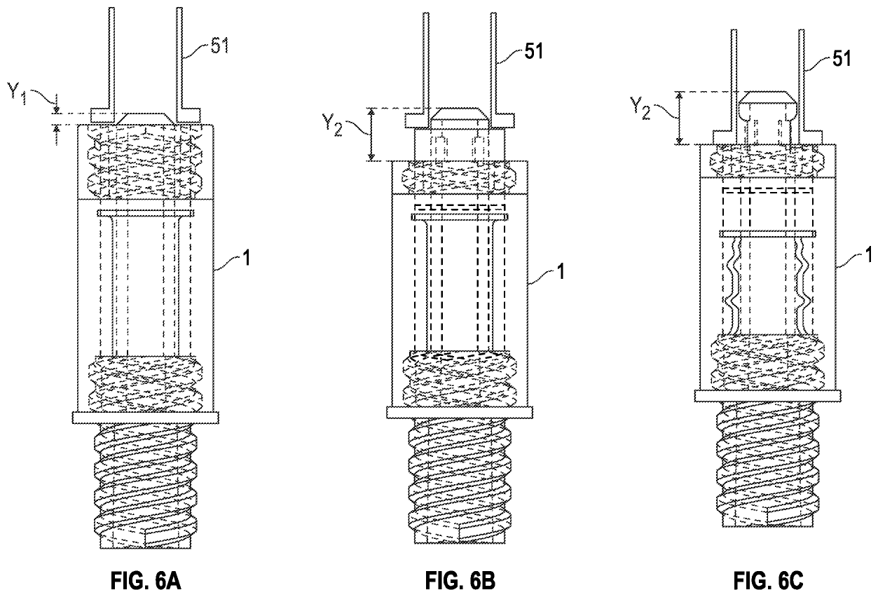

FIGS. 6A-6C also demonstrate a secure connection that avoids leakage issues arising from twist connections. As shown, circumferential wall 14 can comprise internal threads configured to receive an external thread of a female connector, e.g., female connector 51 of a nutrition source, an ISO 80369-3 compliant or compatible connector). A distance Y1 or Y2 may be defined between the internal threads of the circumferential wall and the distal tip of the male conical connector. In certain aspects, an extended distance Y2 may be provided to allow a push fit connector to be connected in a sealed manner to the male conical connector and subsequently secured by twisting the external thread of the female connector 51 within the internal thread of the circumferential wall 14. The push fit connection also may be sealed with respect to the conical tip prior to opening the push valve by push fit connection to the nutrition source connector. In such aspects, advantages of push fit and twist fit connections may both be achieved, without the respective disadvantages.

With a standard distance Y1 as shown in FIG. 6A, the syringe and adapter must first touch and engaged the threads and then be rotated a number of times to secure the recessed conical coupling with the conical tip of the first enteral adapter. In aspects such as shown in FIGS. 6B and 6C comprising a non-standard distance Y2 between the distal end of the male conical tip and the internal threads of the circumferential wall, the external thread of a female connector may contact the nutrition source connector (e.g, male conical tip), form a liquid seal prior to engaging the internal threads of the circumferential wall and without first rotating respective to each other in a leak prone configuration.

For example, FIGS. 6A-6C represent valved embodiments of the present invention configured to receive an external threading of a female connector 51, such as an ISO 80369-3 syringe. In FIG. 6A, standard distance of Y1 causes female connector 51 to first contact and engage the internal threads of circumferential wall 51, and then be rotated a number of times to form a seal. In contrast, FIG. 6B has a distance of Y2 that is greater than Y1. This embodiment demonstrates that with a distance Y2, female connector 51 may contact and form a seal with the nutrition source connector (e.g., male conical tip) without previously engaging threads and without first rotating respective to each other in a leak prone configuration. FIG. 6C also shows an embodiment in which the fluid conduit comprises a push to open valve that is advanced into an open state by the push fit connection, without prior threaded engagement of the female connector to the internal threads of the circumferential wall. Female connector 51 can then be twisted downward to secure the syringe to the enteral adapter while maintaining the push-to-open valve in an open state without risk of leakage.

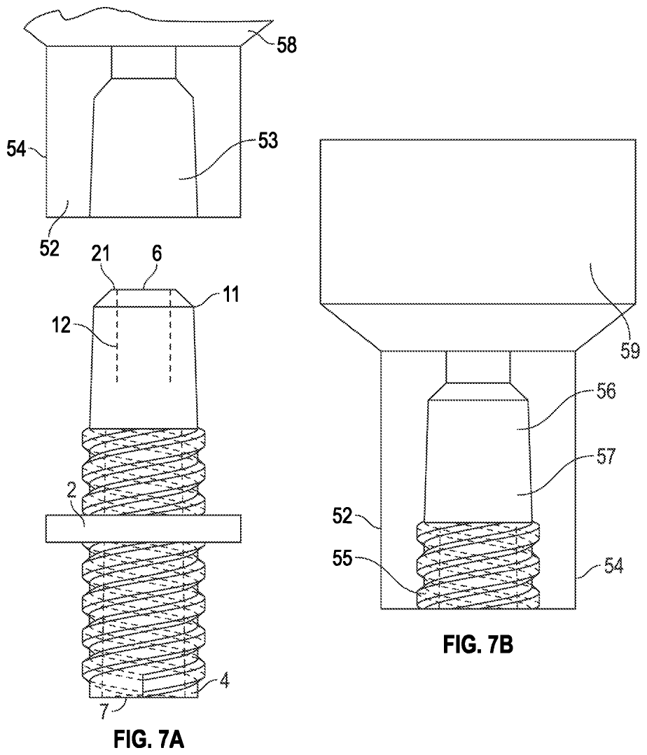

FIG. 7A shows an embodiment of the present invention comprising a “naked” nutrition source connector that can mated with an ISO 80369-3 compliant enteral adapter. This naked adapter has better cleaning capabilities, leakage prevention and infection prevention capabilities than an ISO 80369-3 compliant enteral coupling format. Also shown is a non-ISO 80369-3 connector, female connector 52 comprising a conical recess 53 that can form a push fit and slip over fluid conduit wall 11 of the nutrition source connector. Female connector 52 may be attached to an enteral device such as a syringe, as represented by device 58.

The outer wall 52 of female connector 54 can be formed to various widths so the wall may or may not fit within an ISO 80369-3 connector. The outer wall 52 of female connector 54 can be formed to various widths so the wall may be incompatible with other ISO 80369 connectors that are not compliant with enteral connectors. The outer wall 52 of female connector 54 can be formed to various widths so the wall may be compatible with ISO 80369-3 connectors but incompatible with other ISO 80369 connectors. Enteral adapters as shown in 7A and described herein can facilitate the transition to a new enteral adapter format that can be used with the ISO 80369-3 compliant enteral coupling format, and without the disadvantages of the moat of the ISO 80369-3 compliant enteral coupling format.

FIG. 7B shows an embodiment of the present invention comprising a non-ISO 80369-3 connector. As shown, female connector 54 has a conical recess 57 comprising an internally threaded portion 55 and a non-threaded portion 56. Threaded female connector 54 may be attached to an enteral device such as a syringe, as represented by device 59. In certain aspects, the threaded connector can be compatible with a “naked” enteral connector, for example as shown in FIG. 7A. The combination of these devices may form a new enteral adapter system that provides an enteral adapter system with threaded engagement capability and without the disadvantages of the moat design of the ISO 80369-3 compliant enteral coupling format. The use of enteral adapters in 7A can facilitate the transition to a new enteral adapter format that can be used with the ISO 80369-3 compliant enteral coupling format, and without disadvantages of the ISO 80369-3 compliant enteral coupling format.

Enteral adapters disclosed herein may have a nutrition source connector comprising a conical connector 24 with a threadless circumferential wall 14 surrounding it to provide a surface to seal a cap, or to protect the tip; but without the inner threads that require more manipulation during connection that prolongs the procedure, requires twisting and is more prone to leakage.

Certain aspects of the nutrition source connector may comprise a port such as quick access port without a surrounding collar. The quick access port may have a cap that can form a closed seal of the quick access port without the use of a threaded collar to assist with the seal; or with a cap that would not fit an enteral connector, such as cap with a width greater than the inner threads of an ISO 80369-3 connector.

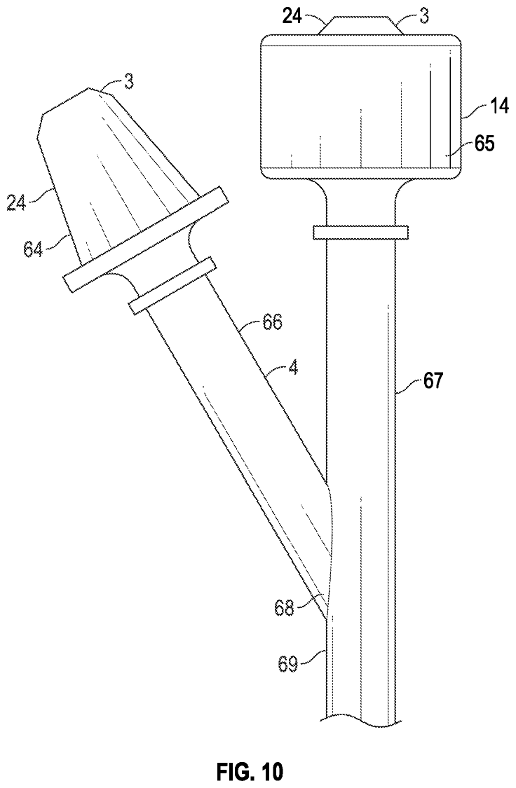

Enteral adapters disclosed herein also can generally comprise any suitable gastrostomy tube connector that allows the enteral adapter to be secured in fluid communication with a feeding tube, either directly as shown in FIG. 10, or indirectly by attachment to a further enteral adapter as shown in the embodiment of FIG. 5E having threadings 45 secured within internal threadings of enteral connector 22.

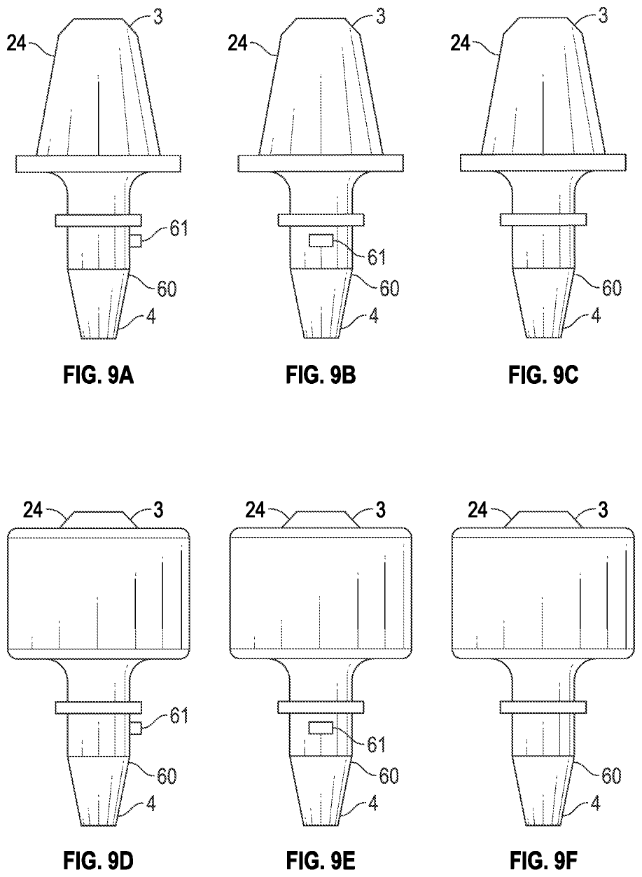

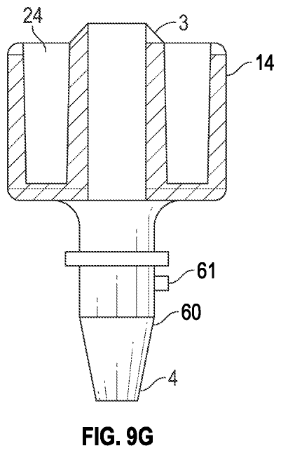

In certain aspects, gastrostomy connectors can be compliant or compatible with the ISO 80369-3 standard for enteral connectors. In certain embodiments, the gastrostomy connector can comprise a mic adapter compatible with a mic-key connector of a feeding tube, for direct transfer of fluid from a feeding device such as an ISO 80369 enter syringe to a gastrostomy tube, such as a low-profile gastrostomy tube without the use of a long intervening flexible tube. FIGS. 9A-H demonstrate an embodiment of an enteral adapter wherein the gastrostomy connector 4 comprises a mic-key compatible mic-key type locking connector 60. As will be understood by those of skill in the art, enteral adapters may generally comprise any combination of gastrostomy connectors and nutrition source connectors suitable for a particular application.

For instance, enteral adapters disclosed herein can comprise a mic-key locking gastrostomy connector and an ISO 80369-3 compliant nutrition source connector. Low profile adapter tips disclosed herein can fit low profile gastrostomy tube adapters. The one end of the embodiment may be the second end of the enteral adapter. Embodiments disclosed herein may be configured to locks in place on one end and provide an ISO 80369-3 compliant or compatible adapter on the other end to facilitate direct boluses of medication or fluid without a bulking long flexible intervening tube that can be difficult to maintain, store, and clean. In certain aspects, gastrostomy connector can comprise an axial projection 61 to engage a gastrostomy tube or gastrostomy tube adapter to facilitate a lock. In certain aspects, projection 61 may be secured within a threading or ridge of a low-profile gastrostomy tube connector by a quarter turn twist lock.

Enteral adapters disclosed herein may therefore be configured so that the gastrostomy connector does not lock in place with a compatible low profile gastrostomy connector on one end for quick access to, and quick disassembly from, a low-profile gastrostomy connector. This mic-key type adapter 60 may have the typical dimensions of a mic-key type locking adapter but without the projection 61 seen in the typical locking type products of the prior art which facilitate a lock such as a quarter turn twist lock with a low-profile gastrostomy tube.

Again, enteral adapters as disclosed herein may comprise any combination of nutrition source adapters and gastrostomy connectors as disclosed herein. In certain aspects, a male conical tip 24 can be incorporated in “naked” fashion, lacking a circumferential wall, as shown in FIGS. 9A-9C. Alternatively, nutrition source connector can comprise a circumferential wall 14, either with internal threadings, or without internal threadings as shown in FIGS. 9D-9H.

In certain aspects, enteral adapters may comprise a gastrostomy connector as shown in FIGS. 9A-H, paired with an ISO 80369-3 compliant or compatible nutrition source connector (e.g., a male ISO80369-3 coupling) on an opposing end of the adapter to facilitate a more stable a connection with an enteral syringe and direct boluses of medication or fluid without a bulking long flexible intervening tube that can be difficult to maintain, store and clean. The use of such enteral adapters would remove disadvantages of using tubes in such situations and would remove the need to lock and unlock a connector for a quick low volume bolus of medication. This would reduce the twisting and shearing applied to the skin with such connections.

In certain aspects, enteral adapters disclosed herein may be monolithic to increase stiffness, reduce choking hazards, or simplify manufacturing.

### Enteral Feeding Systems

Enteral feeding systems incorporating enteral adapters disclosed above are also contemplated herein. Generally, enteral feeding systems disclosed herein can provide advantages to ease of connection, speed of delivery, leak prevention, maintaining a sanitary condition of the any or all components of the enteral feedings system (e.g., feeding tube, feeding tube connector, enteral adapter, nutrition source), and ease of cleaning components of the enteral feeding system. Enteral feeding systems disclosed herein may exhibit such advantages while maintaining compliance and/or compatibility with existing and applicable standards (e.g., ISO 80369-3 connectors). Systems disclosed herein also may be incompatible with other connectors, for instance to prevent misconnections between IV sources and feeding tube connectors.

Enteral feedings systems can comprise an enteral adapter as disclosed herein, and a nutrition source configured to connect to the nutrition source connector of the enteral adapter. In certain aspects, the nutrition source is a single-use nutrition source. In certain aspects, the nutrition source can be a food pouch. In certain aspects, the nutrition source can be a syringe (e.g., an enteral syringe). FIG. 7A provides an embodiment of a nutrition source 58 configured to connect to the nutrition source connector of an enteral adapter at fluid conduit wall 11. FIG. 7B demonstrates a further example of a nutrition source comprising a threaded connection capable of securing to the external threads of the enteral adapter shown in FIG. 7A. Combination of either enteral syringe 58 or enteral syringe 59 and enteral adapters may be further connected to a feeding tube at the gastrostomy connector 4 of the enteral adapter, such that the nutrition source becomes engaged in fluid communication with the feeding tube.

In certain aspects, the nutrition source comprises a female enteral connector with external threads and an opening larger than a female Luer lock connector to provide improved fluid flow and prevent misconnections. In other aspects, the nutrition source can comprise an ISO 80369-3 connector. In further aspects, the nutrition source can comprise an internally threaded portion configured to connect to the nutrition source connector as shown in FIG. 7B. Alternatively, the nutrition source comprises an externally threaded portion configured to connect to the nutrition source connector.

Certain embodiments disclosed herein may have multiple enteral adapters as shown by FIG. 10, to provide multiple types of functions not provided by a single adapter type. Embodiments may have multiple adapters of different types at one end of a gastrostomy tube such as 69, at least one compliant adapter such as could be represented by adapter 65 and at least one non-compliant adapter such as could be represented by 64. Embodiments may at least one removable collar and at least one non-removable collar at each end; at least one non-threaded connector at an end; at least one valve and one non-valved connector at each end; a quick slip fit connector adapter as could be represented by 64 and a more secured twist fit connector such as could be represented by 65; a branched adapter; a y-adapter 68 with multiple adapter types; and/or a w-adapter with multiple adapter types and multiple fluid conduits such as 66 and 67 that converge into a single fluid conduit, such as 69.

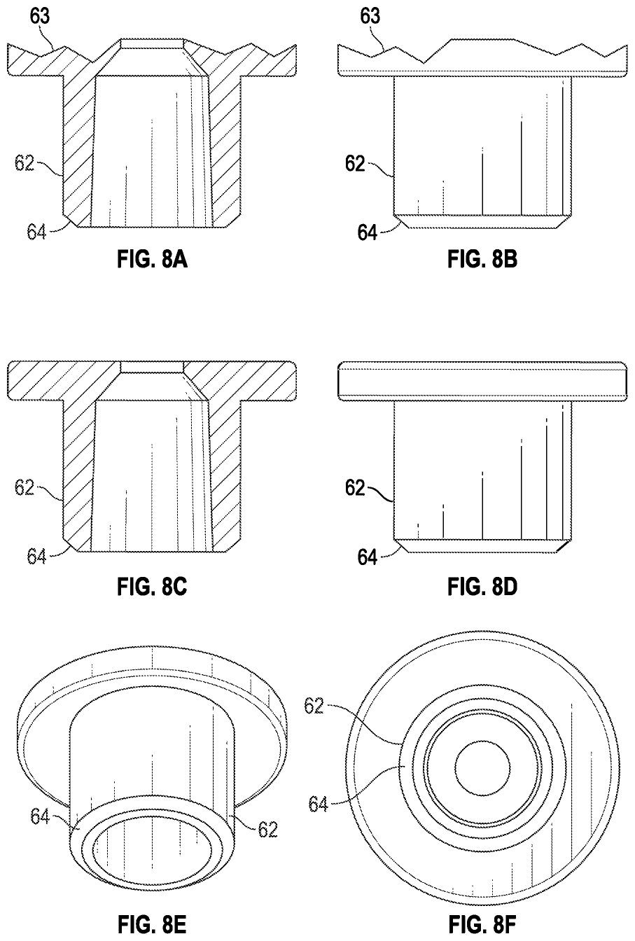

Enteral feeding systems can further comprise accessories to facilitate the assembly, disassembly, mobility, and ease of use and transport of the system. For instance, in certain aspects, enteral feeding systems can further comprise a carrying case. In other aspects, the enteral feeding system can further comprise a cleaning solution or sanitary wipe. Other aspects of enteral feeding systems can comprise any or all of a cap for the nutrition source connector of the enteral adapter, a cap for the nutrition source, and a cap for the feeding tube connector. FIGS. 8C-F provide cap embodiments configured to mate with an ISO 80369-3 compliant or compatible connector, as distinguished by the width of connector walls 62.

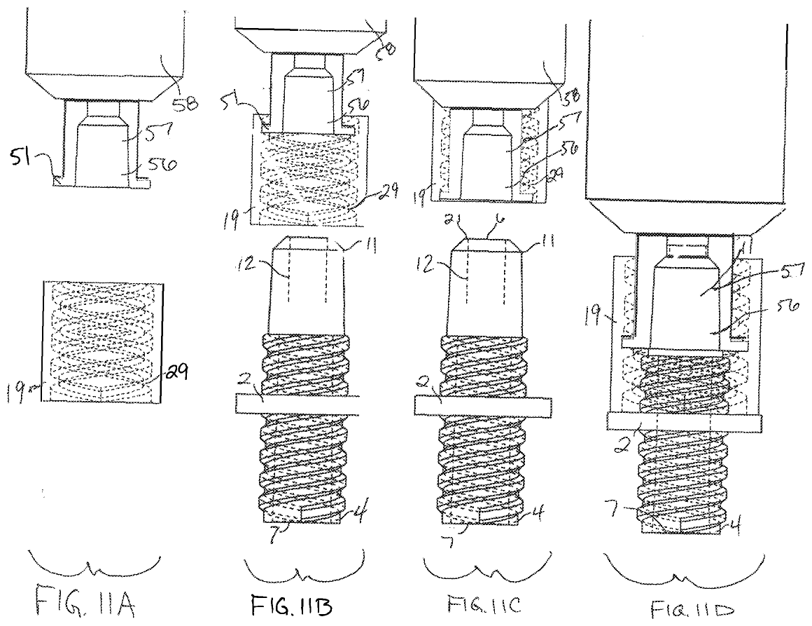

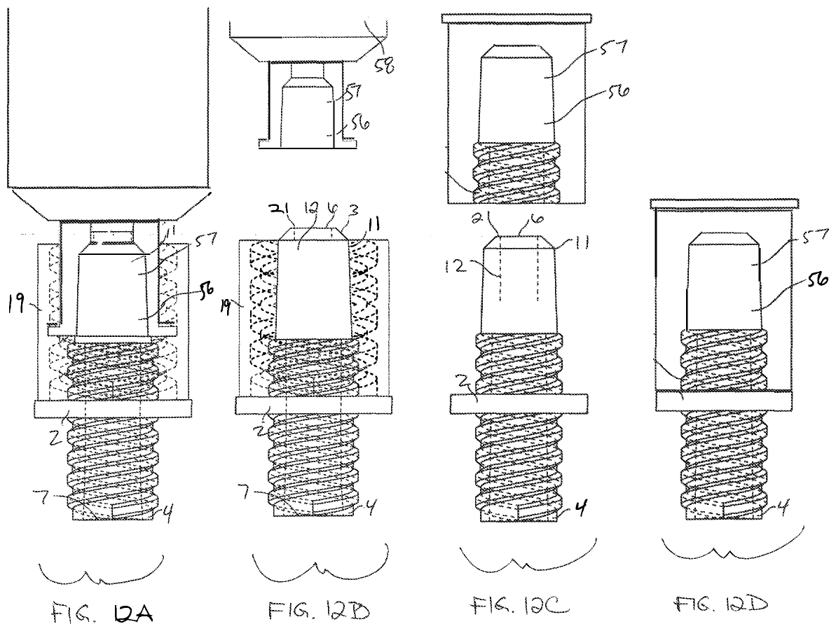

FIGS. 11A-11D demonstrate another example of an enteral feeding system contemplated herein. As shown, certain embodiments of the enteral feeding system disclosed herein can comprise an enteral adapter generally as described above in FIG. 3A and a circumferential wall for securing an enteral syringe to the adapter. FIG. 11A provides an embodiment of an enteral syringe with a female Luer-lock type connector, e.g., having a leading rim configured to interface with the internal threads of circumferential wall 19. In this manner, the circumferential wall can be first attached to the enteral syringe to allow a push fit connection to the enteral adapter prior to advancing the circumferential wall along the threading of the female Luer lock and securing to the external threadings of the enteral base. In other aspects, the circumferential wall may be first attached to, or provided with, the enteral adapter to reduce the number of steps to attachment of the enteral syringe (e.g., a one-step twist fit connection of the enteral syringe within the circumferential wall. However, it is unexpectedly found that the cleanliness of the connection may be improved by first completing a push fit connection prior to securing the connection by advancing the circumferential wall about the connection. In this manner, the advantages of both a push fit connection (e.g., a quick and complete leak-proof attachment with pressure applied during connection when manual pressure is being applied) and a twist fit connection (e.g., a secure and leak-proof connection after connection, when manual pressure is no longer being applied) can be realized within a single enteral feeding system, while also improving the convenience and accessibility of the attachment methodology.

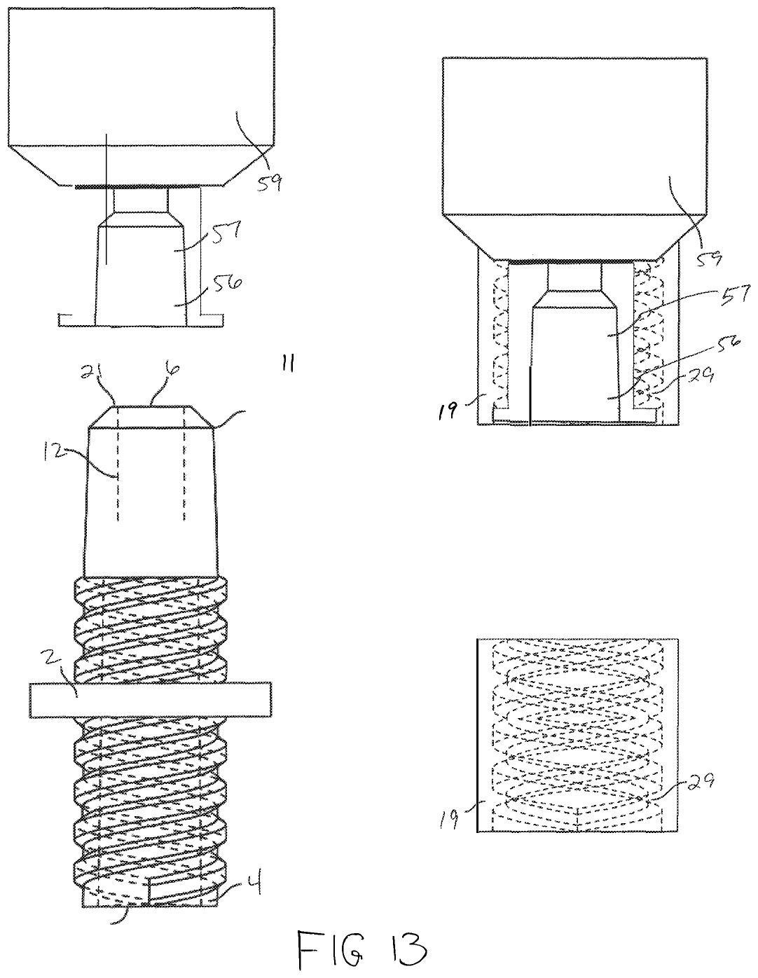

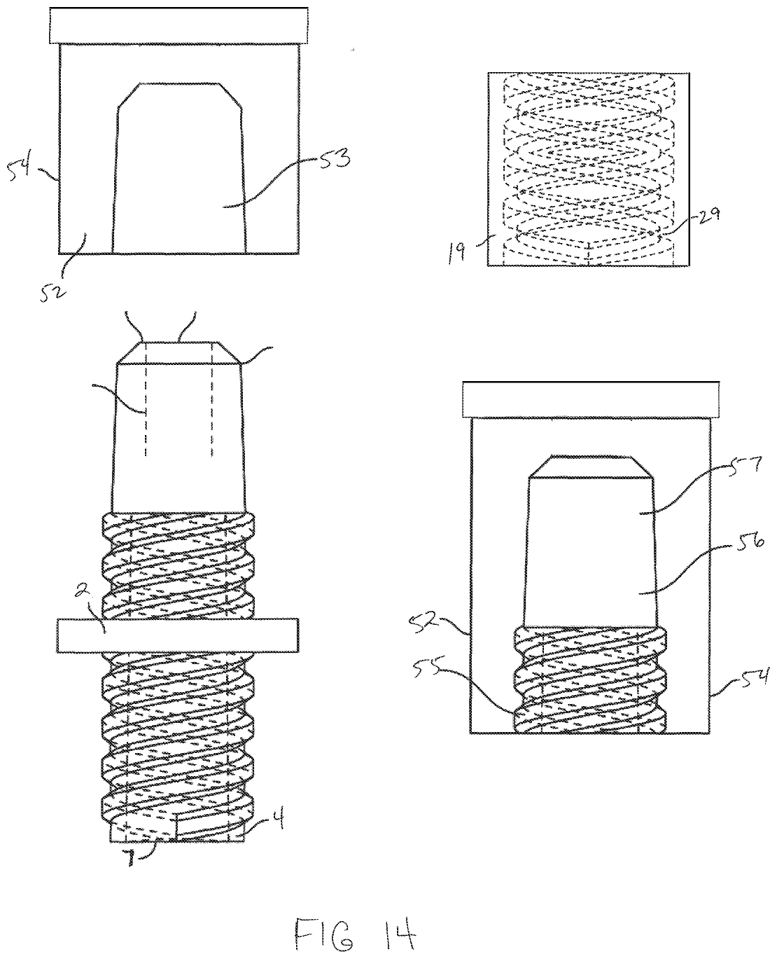

FIGS. 12A-12D show the disconnection sequence of the enteral feeding system, generally as may follow the connection sequence for enteral feeding systems as described in FIGS. 11A-11D. FIG. 12A is a reproduction of FIG. 11D, and shown relative to FIG. 12B to demonstrate a first disconnection step. As shown in FIG. 12B, the enteral syringe may be removed from circumferential wall 19 after delivering the nutritional substance to the gastrostomy tube, leaving the collar in place on the enteral adapter. Alternatively, and in reverse of the sequential push-fit then twist-fit method described directly above, the circumferential wall may be first advanced onto the enteral syringe to interrupt the twist fit connection between the syringe and the adapter, prior to interrupting the push fit connection between the syringe and the adapter.

In either disconnection method, the circumferential wall may be removed and/or discarded from the enteral adapter (or the enteral syringe), and a cap may be provided and installed on the enteral adapter to protect the gastrostomy tube and enteral adapter connected thereto from contamination when the gastrostomy tube is not in active use. FIG. 12C shows the enteral adapter having the enteral syringe disconnected and the circumferential wall removed, now provided with a cap for twist fit connection to the external threadings of the enteral adapter. In certain embodiments, the cap can be secured to the enteral adapter by push fit connection. FIG. 12D shows the cap secured to the enteral adapter by twist-fit connection in a capped arrangement.

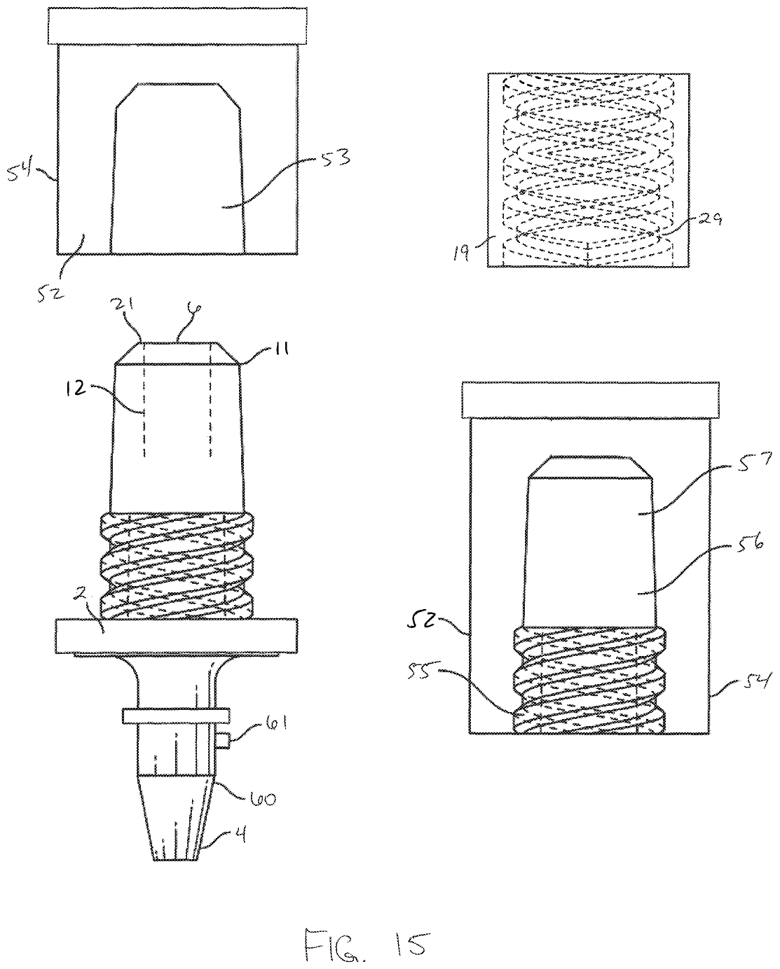

FIGS. 13-15 provide further examples of enteral feeding systems as kits that may be assembled and provided as several interacting and compatible components. FIG. 13 provides, for example, an enteral adapter, an enteral syringe comprising a female connector 51, and a circumferential wall 19. As shown, circumferential wall 19 may be secured to the female connector 51 of the enteral syringe during assembly, storage, and during push-fit connection to the enteral adapter as shown in FIGS. 11A-11D.

FIG. 14 provides another example of a kit comprising and enteral adapter and provided with either or both of an internally threaded cap or a push-fit cap configured to connect to the enteral adapter. The kit of FIG. 14 also can comprise a circumferential wall 19 that may be alternately secured to the enteral adapter prior to connection to an enteral syringe or another nutrition source separately provided. FIG. 15 provides yet another example of a kit comprising an enteral adapter and either or both of an internally threaded cap and a push-fit cap. The enteral adapter of FIG. 15 is shown as having a mic-key connection to a gastrostomy tube as opposed to the externally threaded connector of FIG. 14. Those of skill in the art will understand that the enteral adapter connection to the gastrostomy tube can be any disclosed herein, or commonly known, as may be appropriate to allows the user to realize the benefits of enteral adapters, enteral feeding systems, kits, and methods disclosed herein.

### Methods for Introducing Nutritional Substances to a Gastrostomy Tube

Methods for using enteral adapters and enteral feeding systems disclosed above are also contemplated herein. Particularly, methods for providing nutrition from a nutrition source into a gastrostomy tube are contemplated herein with improved efficiency and ease. Methods disclosed herein may also allow for easier maintenance, reduced risk contamination, and faster applications. In certain aspects, the risk of introducing bacteria into the gastrostomy tube is reduced (e.g., by at least 10%, by at least 20%, by at least 50%) compared to an identical process connecting the nutrition source directly to a gastrostomy tube comprising an ENFit® connector. Leakage also may be improved, as described above, typically resulting in reduced biocontamination, mess, waste, unreliable dosing and other associated problems.

Generally, methods disclosed herein can comprise connecting an enteral adapter as described herein to a gastrostomy tube, connecting a nutrition source to the enteral adapter, transferring a substance from the nutrition source to the gastrostomy tube through the enteral adapter, and disconnecting the enteral adapter from the gastrostomy tube. As stated above, the transferred substance need not be any particular type of nutrient or medicinal substance, and generally may be any commonly applied by gastrostomy tube, as will be understood in the art. However, methods disclosed herein can comprise transferring a more concentrated thick or viscous substance than might otherwise be possible where fluid conduit sizes are increased relative to the allowable width under the ISO 80369-3 standard. Feeds performed according to methods herein therefore may be faster and less prone to clogging, particularly compared to ISO 80369-3 connectors. Methods disclosed herein also may require less blending, and therefore nutritious substance can be prepared without specialized equipment and high-end blenders.

In certain aspects, methods can comprise discarding either or both of the enteral adapter and the nutrition source. It follows that components of the enteral feeding systems disclosed above may be disposable, or reusable. For instance, where an enteral adapter becomes thoroughly soiled or damaged, the enteral adapter may be discarded and easily replaced without having to replace the feeding tube or repair a connection to the feeding tube.

Methods contemplated herein can comprise connecting components of enteral feeding systems and adapters in particular order to reduce leaking. For instance, methods can comprise securing a circumferential wall to the nutrition source, and then securing the nutrition source to the enteral adapter, and then securing the circumferential wall to the enteral adapter. In this fashion, the circumferential wall can serve as a twist-fit connection that follows the push-fit connection to retain advantages of both without respective disadvantages. Such aspects may reduce disconnections, maintain connections for a prolonged feeding schedule and provide easier, safer, more convenient access for frequent non-prolonged interval feeding schedules.

Similarly, certain methods can further comprise removing or retracting a circumferential collar surrounding the nutrition source connector, for instance after transferring the substance to the feeding tube to clean the enteral adapter or the circumferential collar. In other aspects, for instance where a circumferential collar is disposable, methods can comprise removing or retracting a first internally threaded circumferential collar surrounding the nutrition source connector, cleaning the nutrition source connector, positioning a second internally threaded circumferential collar around the nutrition source connector.

The invention is described herein with reference to numerous aspects and specific examples. Many variations will suggest themselves to those skilled in the art in light of the detailed description. All such obvious variations are within the full intended scope of the appended claims. Other aspects of the invention can include, but are not limited to, the following (aspects are described as “comprising” but, alternatively, can “consist essentially of” or “consist of”):

### ASPECTS

Aspect 1. An enteral adapter comprising:

a nutrition source connector;

a gastrostomy tube connector; and

a fluid conduit extending from the nutrition source connector to the gastrostomy tube connector;

wherein the nutrition source connector is a push fit connector.

Aspect 2. The adapter of aspect 1, wherein the push fit connector comprises a conical tip.

Aspect 3. The adapter of aspect 2, wherein the fluid conduit extends to an opening in the conical tip.

Aspect 4. The adapter of aspect 2, wherein the opening in the conical tip is in a tapered surface of the conical tip.

Aspect 5. The adapter of any one of aspects 2-4, further comprising a circumferential wall surrounding the conical tip.

Aspect 6. The adapter of aspect 5, wherein the circumferential wall is retractable from a closed position blocking flow into the fluid conduit to an open position.

Aspect 7. The adapter of aspect 6, wherein the circumferential wall is attached to the adapter by a silicon sleeve.

Aspect 8. The adapter of aspect 6, wherein the circumferential wall is removable.

Aspect 9. The adapter of any one of aspects 5-8, further comprising a second circumferential wall.

Aspect 10. The adapter of aspect 6, wherein the second circumferential wall is removable.

Aspect 11. An enteral adapter comprising:

a nutrition source connector comprising a conical tip;

a gastrostomy tube connector;

a fluid conduit extending from an opening in the nutrition source connector to an opening in the gastrostomy tube connector; and

a circumferential wall surrounding at least a portion of the conical tip, wherein the circumferential wall is removable or retractable.

Aspect 12. The adapter of aspect 11, wherein the circumferential wall is removable from the adapter.

Aspect 13. The adapter of aspect 12, wherein at least a portion of the circumferential wall comprises internal threads.

Aspect 14. The adapter of aspect 13, wherein the internal threads of the circumferential wall are configured to receive an external thread of a female connector.

Aspect 15. The adapter of aspect 13 or 14, wherein the internal threads of the circumferential wall extend at least from a distal portion of the circumferential wall to the base of the conical tip when secured to the adapter.

Aspect 16. The adapter of any one of aspects 13-15, wherein at least a portion of the internal threads of the circumferential wall are configured to mate with external threads of the nutrition source connector.

Aspect 17. The adapter of aspect 13, wherein the nutrition source connector comprises an externally threaded surface proximal to the conical tip, the external threads configured to mate with the internal threads of the circumferential wall.

Aspect 18. The adapter of aspect 17, wherein the external threads of the nutrition source connector extend from the base of the conical tip.

Aspect 19. The adapter of aspect 11, wherein the circumferential wall is retractable to a position that does not partially surround the conical tip.

Aspect 20. The adapter of aspect 19, wherein the circumferential wall comprises internal threads configured to receive an external thread of a female connector.

Aspect 21. The adapter of aspect 20, wherein the nutrition source connector comprises a partition at the proximal end of the conical tip, the partition securing an axial position of the circumferential wall when the external thread of the female connector is secured within the internal threads of the circumferential wall.

Aspect 22. The adapter of aspect 20 or 21, wherein the female connector is an ISO 80369-3 connector.

Aspect 23. The adapter of any one of aspects 20-22, further comprising a partition between the nutrition source connector and the gastrostomy tube connector configured to retain the circumferential wall in an axial relationship with the adapter when the circumferential wall is retracted from surrounding the conical tip.

Aspect 24. An enteral adapter comprising:

a nutrition source connector;

a gastrostomy tube connector; and

a fluid conduit extending from an opening in the nutrition source connector to an opening in the gastrostomy tube connector;

wherein the opening in the nutrition source connector comprises a valve configured to alternatively open and close the fluid conduit.

Aspect 25. The adapter of aspect 24, wherein the valve is configured to move from a closed position to an open position by connection of a nutrition source to the nutrition source connector.

Aspect 26. The adapter of aspect 24 or 25, wherein the valve is a twist-to-open valve.

Aspect 27. The adapter of any one of aspects 24-26, wherein the nutrition source connector comprises a conical tip.

Aspect 28. The adapter of aspect 27, further comprising a circumferential wall at least partially surrounding the conical tip.

Aspect 29. The adapter of aspect 28, wherein the circumferential wall comprises internal threads configured to receive an externally threaded female connector.

Aspect 30. The adapter of any one of aspects 27-29, wherein the conical tip comprises a stem that extends axially from the conical tip when moved from the closed position to the open position.

Aspect 31. The adapter of aspect 25, wherein the valve is a push-to-open valve.

Aspect 32. The adapter of aspect 31, wherein the nutrition source connector comprises a conical tip.

Aspect 33. The adapter of aspect 32, wherein the valve comprises a circumferential wall obstructing an opening in the conical tip in the closed position.

Aspect 34. The adapter of aspect 33, wherein the circumferential wall is axially retractable from the opening in the conical tip.

Aspect 35. The adapter of aspect 34, wherein the circumferential wall comprises a compressible silicon sleeve secured to the adapter.

Aspect 36. The adapter of any one of aspects 31-35, further comprising a second circumferential wall.

Aspect 37. The adapter of any one of aspects 1-36, wherein the gastrostomy tube connector is capable of connecting to an ISO 80369-3 connector.

Aspect 38. The adapter of aspect 37, wherein the gastrostomy tube connector is configured to connect to a key lock gastrostomy valve.

Aspect 39. The adapter of aspect 37, wherein the gastrostomy tube connector is an ISO 80369-3 compliant threaded connector configured to engage threads of a male ISO 80369-3 connector.

Aspect 40. The adapter of aspect 39, wherein the gastrostomy tube connector comprises an axial projection configured to interact with threadings of the male ISO 80369-3 connector.

Aspect 41. The adapter of any one of aspects 1-40, wherein the nutrition source connector is capable of connecting to a standard externally threaded ISO 80369-3 syringe.

Aspect 42. The adapter of any one of aspects 1-41, wherein the nutrition source connector is incompatible with a standard female Luer connector.

Aspect 43. An enteral feeding system comprising:

the enteral adapter of any one of aspects 1-42; and

a nutrition source configured to connect to the nutrition source connector of the enteral adapter.

Aspect 44. The system of aspect 43, wherein the nutrition source comprises a female enteral connector with external threads and an opening larger than a female Luer lock connector.

Aspect 45. The system of aspect 43, wherein the nutrition source comprises an ISO 80369-3 connector.

Aspect 46. The system of aspect 43, wherein the nutrition source comprises an internally threaded portion configured to connect to the nutrition source connector.

Aspect 47. The system of aspect 43, wherein the nutrition source comprises an externally threaded portion configured to connect to the nutrition source connector.

Aspect 48. The system of any one of aspects 43-47, wherein the nutrition source is a syringe.

Aspect 49. The system of any one of aspects 43-47, wherein the nutrition source is a food pouch.

Aspect 50. The system of any one of aspects 43-49, further comprising a carrying case.

Aspect 51. The system of any one of aspects 43-50, further comprising cleaning solution.

Aspect 52. The system of any one of aspects 43-51, further comprising sanitary wipes.

Aspect 53. The system of any one of aspects 43-52, wherein the nutrition source is a single-use nutrition source.

Aspect 54. The system of any one of aspects 43-53, further comprising a cap for the nutrition source connector of the enteral adapter.

Aspect 55. A method for providing nutrition from a nutrition source into a gastrostomy tube, the method comprising:

connecting the enteral adapter of any one of aspects 1-42 to a gastrostomy tube;

connecting the nutrition source to the enteral adapter;

transferring a substance from the nutrition source to the gastrostomy tube through the enteral adapter;

disconnecting the enteral adapter from the gastrostomy tube.

Aspect 56. The method of aspect 55, further comprising discarding either or both of the enteral adapter and the nutrition source.

Aspect 57. The method of aspect 55, further comprising removing or retracting a circumferential collar surrounding the nutrition source connector.

Aspect 58. The method of aspect 55, further comprising cleaning the enteral adapter.

Aspect 59. The method of aspect 55, further comprising removing or retracting a first internally threaded circumferential collar surrounding the nutrition source connector; cleaning the nutrition source connector; positioning a second internally threaded circumferential collar around the nutrition source connector.

Aspect 60. The method of any one of aspects 55-60, wherein a risk of introducing bacteria into the gastrostomy tube is reduced (e.g., by at least 10%, by at least 20%, by at least 50%) compared to an identical process connecting the nutrition source directly to a gastrostomy tube comprising an ENFit® connector.

1. An enteral adapter comprising:
a nutrition source connector;
a gastrostomy tube connector; and
a fluid conduit extending from the nutrition source receiving connector to the gastrostomy tube delivery connector;
wherein the nutrition source connector is a push fit connector.

2. The adapter of claim 1, wherein the nutrition source connector is a push fit connector that permits a connection to a standard externally threaded female ISO 80369-3 compliant syringe without twisted engagement.

3. The adapter of claim 1, wherein an external profile of the nutrition source connector does not match an external profile of a male conical tip of an ISO 80369-3 compliant connector.

4. The adapter of claim 1, wherein the nutrition source connector comprises a valved connector.

5. The adapter of claim 1, wherein the nutrition source connector comprises an obstructed central axis.

6. The adapter of claim 1, wherein the enteral adapter comprises an externally threaded surface between a male conical tip of the nutritional source connector and the gastrostomy tube connector.

7. The adapter of claim 1, gastrostomy tube connector comprises a key lock projection configured to fit a key lock receiving connector of a gastrostomy tube.

8. The adapter of claim 1, further comprising a circumferential wall surrounding at least a portion of the nutrition source connector, wherein the circumferential wall is removable or retractable, and wherein an internally threaded surface of the circumferential wall is capable of mating with an externally threaded female ISO 80369-3 connector.

9. The adapter of claim 1, wherein the nutrition source connector comprises a male conical tip, and wherein an external profile of the male conical tip is compliant with ISO 80369-3.

10. An enteral adapter comprising:
a nutrition source connector comprising a conical tip and a circumferential wall surrounding at least a portion of the conical tip;
a gastrostomy tube connection; and
a fluid conduit extending from the nutrition source connection to the gastrostomy tube connector;
wherein the circumferential wall is removeable or retractable.

11. The adapter of claim 10, wherein the circumferential wall is removable.

12. The adapter of claim 10, wherein:
the circumferential wall comprises internal threads configured to receive an externally threaded wide based female connector with a conical recess;
the internal threads are incompatible with a female Luer lock base; and
the internal threads are configured to receive an externally-threaded female ISO 80369-3 connector.

13. An enteral adapter comprising:
a nutrition source connector;
a gastrostomy tube connector; and
a fluid conduit extending from the nutrition source connector to the gastrostomy tube connector;
wherein the nutrition source connector comprises a valve configured to alternatively open and close the fluid conduit.

14. The adapter of claim 13, wherein the valve is configured to move from a closed position to an open position by connection of a nutrition source to the nutrition source connector.

15. The adapter of claim 14, wherein the valve is a twist-to-open valve.

16. The adapter of claim 15, further comprising a circumferential wall at least partially surrounding the conical tip; wherein the circumferential wall comprises internal threads configured to receive a female ISO 80369-3 connector.

17. The adapter of claim 14, wherein the valve is a push-to-open valve.

18. The adapter of claim 17, further comprising a retractable circumferential wall comprising a compressible sleeve.

19. An enteral feeding system comprising:
the enteral adapter of claim 1;
a nutrition source configured to connect to the nutrition source connector of the enteral adapter; and
a gastrostomy tube comprising an ISO 80369-3 connection.

20. A method for providing a nutritious substance from a nutrition source into a gastrostomy tube, the method comprising:
connecting the enteral adapter of claim 1 to a gastrostomy tube;
connecting the nutrition source to the enteral adapter;
transferring the nutritious substance from the nutrition source to the gastrostomy tube through the enteral adapter; and
disconnecting the enteral adapter from the gastrostomy tube.
